SEQUENCE LISTING (14 sequences)

SEQ ID NO: 1 - protein, 9 aa, Artificial Sequence
  Synthetic construct
CGNKRTRGC

SEQ ID NO: 2 - protein, 7 aa, Artificial Sequence
  Synthetic construct
GNKRTRG

SEQ ID NO: 3 - protein, 6 aa, Artificial Sequence
  Synthetic construct
CLSDGK

SEQ ID NO: 4 - protein, 9 aa, Artificial Sequence
  Synthetic construct
CLSDGKRKC

SEQ ID NO: 5 - protein, 9 aa, Artificial Sequence
  Synthetic construct
CLSDGKPVS

SEQ ID NO: 6 - protein, 7 aa, Artificial Sequence
  Synthetic construct
CASLSCR

SEQ ID NO: 7 - protein, 9 aa, Artificial Sequence
  Synthetic construct
CLDGGRPKC

SEQ ID NO: 8 - protein, 9 aa, Artificial Sequence
  Synthetic construct
CREAGRKAC

SEQ ID NO: 9 - protein, 9 aa, Artificial Sequence
  Synthetic construct
CSMSAKKKC

SEQ ID NO: 10 - protein, 9 aa, Artificial Sequence
  Synthetic construct
CKTRVSCGV

SEQ ID NO: 11 - protein, 9 aa, Artificial Sequence
  Synthetic construct
CAGRRSAYC

SEQ ID NO: 12 - protein, 9 aa, Artificial Sequence
  Synthetic construct
CSGGKVLDC

SEQ ID NO: 13 - protein, 9 aa, Artificial Sequence
  Synthetic construct
CNRRTKAGC

SEQ ID NO: 14 - protein, 9 aa, Artificial Sequence
  Synthetic construct
CNKRTRGGC

(19) United States
(12) Patent Application Publication
(10) Pub. No.: US 2011/0311616 A1
(43) Pub. Date: Dec. 22, 2011

(54) TARGETING TUMOR ASSOCIATED MACROPHAGES USING BISPHOSPHONATE-LOADED PARTICLES

(76) Inventors: Jeff Smith, San Diego, CA (US); Gaurav Sharma, San Diego, CA (US)

(21) Appl. No.: 13/162,419

(22) Filed: Jun. 16, 2011

Related U.S. Application Data
(60) Provisional application No. 61/355,968, filed on Jun. 17, 2010.

(51) Int. Cl. A61K 31/663; A61P 35/00; A61K 9/14; A61K 9/127; B82Y 5/00

(52) U.S. Cl. 424/450; 424/490; 424/491; 514/102; 514/108; 977/773; 977/906; 977/907

(57) ABSTRACT

Provided herein are compositions including particles that comprise a bisphosphonate compound and a tumor specific targeting peptide or peptidomimetic. Also provided herein are methods of reducing tumor associated macrophages and methods of treating cancer with said compositions.

### CROSS-REFERENCE TO RELATED APPLICATIONS

This application claims priority under 35 U.S.C. §119(e) to U.S. Provisional Patent Application Ser. No. 61/355,968, filed on Jun. 17, 2010, which is incorporated herein by reference in its entirety.

### REFERENCE TO SEQUENCE LISTING

The present application is being filed along with a Sequence Listing in electronic format. The Sequence Listing is provided as a file entitled BURNHAM.033A, created Jun. 16, 2011, which is 2.76 KB in size. The information in the electronic format of the Sequence Listing is incorporated herein by reference in its entirety.

### FIELD OF THE INVENTION

This application relates generally to the fields of cancer biology and drug delivery. More particularly, embodiments provided herein are drawn to particles comprising a bisphosphonate compound a tumor specific targeting peptide or peptidomimetic useful for reducing macrophage density in a tumor and treating cancer.

### BACKGROUND

Tumor associated macrophages (TAMs) are known to be important for tumor growth. TAMs originate from circulating monocytes and their recruitment into tumors is driven by tumor-derived chemotactic factors. TAMs promote tumor cell proliferation and metastasis by secreting a wide range of growth and proangiogenic factors. Consequently, many tumors with a high number of TAMs have an increased tumor growth rate, local proliferation and distant metastasis. In fact, the extent of TAM infiltration has been used as an inverse prognostic predictor in breast cancer, head and neck cancer, prostate and uterine cancer (R. D. Leek, R. Landers, S. B. Fox, F. Ng, A. L. Harris, C. E. Lewis, British journal of cancer 1998, 77, 2246; M. R. Young, M. A. Wright, Y. Lozano, M. M. Prechel, J. Benefield, J. P. Leonetti, S. L. Collins, G. J. Petruzzelli, International Journal of Cancer 1997, 74, 69; I. F. Lissbrant, P. Stattin, P. Wikstrom, J. E. Damber, L. Egevad, A. Bergh, International journal of oncology 2000, 17, 445; H. B. Salvesen, L. A. Akslen, International Journal of Cancer 1999, 84, 538). TAMs are also prominent in tumor tissues, comprising up to 80% of the cell mass in breast carcinoma.

Therapeutic targeting of TAMs is still in its infancy. Bisphosphonate compounds can be used to selectively induce TAM apoptosis, but current approaches for attacking TAMs are ineffective and unsuitable because such approaches are toxic toward all macrophages, not just TAMs, and this non-specificity poses serious side effects.

### SUMMARY OF THE INVENTION

In one embodiment, a composition is provided including a particle which comprises a bisphosphonate compound and a tumor specific targeting peptide or peptidomimetic bound to a surface on the particle. In one aspect, the particle is a nanoparticle. In the same aspect, the nanoparticle comprises a polymer. Further in the same aspect, the polymer is poly(lactide-co-glycolide) (PLGA). Additionally in the same aspect, the bisphosphonate compound is clodronate. Further in the same aspect, the peptide or peptidomimetic comprises the amino acid sequence of SEQ ID NO:1. In another aspect, the particle is a liposome. In the same aspect, the bisphosphonate compound is clodronate. Further in the same aspect, the peptide or peptidomimetic comprises the amino acid sequence of SEQ ID NO:1.

In another embodiment, provided is a method of reducing tumor associated macrophage density in a tumor of a subject comprising administering to a subject having a tumor an effective amount of a plurality of particles comprising a bisphosphonate compound and a tumor specific targeting peptide or peptidomimetic bound to a surface on the particles, wherein the effective amount of the particles is sufficient to reduce the density of tumor associated macrophages in the tumor of the subject. In one aspect, the particle is a nanoparticle comprising a polymer. In the same aspect, the polymer comprises poly(lactide-co-glycolide) (PLGA). Further in the same aspect, the bisphosphonate compound is clodronate. Additionally in the same aspect, the peptide or peptidomimetic comprises the amino acid sequence of SEQ ID NO:1.

In an additional embodiment, provided is a method of treating cancer in a subject comprising administering to a subject having cancer an effective amount of a plurality of particles comprising a bisphosphonate compound and a tumor specific targeting peptide or peptidomimetic bound to a surface on the particles, wherein the effective amount of the particles is sufficient to treat the cancer. In one aspect, the particle is a liposome. In the same aspect, the particle is a nanoparticle comprising a polymer. In the same aspect, the polymer is poly(lactide-co-glycolide) (PLGA). Further in the same aspect, the bisphosphonate molecule is clodronate. Additionally in the same aspect, the peptide or peptidomimetic comprises the amino acid sequence of SEQ ID NO:1.

### BRIEF DESCRIPTION OF THE DRAWINGS

FIG. 1 is a schematic illustration of the fabrication of LyP-1 functionalized PLGA nanoparticles for tumor targeting.

FIG. 2 shows physical and kinetic properties of clodronate-loaded PLGA nanoparticles. FIG. 2a is a scanning electron microscope image of clodronate-loaded PLGA nanoparticles. FIG. 2b is a dynamic light scattering histogram showing the size distribution of clodronate-loaded PLGA nanoparticles. FIG. 2c is a graph showing the drug release kinetics of clodronate-loaded nanoparticles in vitro.

FIG. 3 shows selective toxicity of clodronate-loaded nanoparticles in vitro. FIG. 3a is a graph showing the in vitro dose dependent toxicity of clodronate-loaded nanoparticles on RAW264.7 macrophages. FIG. 3b is a graph showing that clodronate-loaded nanoparticles are not toxic to the non-phagocytic CCL-110 fibroblast cell line.

FIG. 4 is a panel of fluorescent microscopic images showing that LyP-1 functionalized nanoparticles selectively target tumor associated macrophages. FIG. 4, top row, shows staining of 4T1 tumors treated with LyP-1 functionalized nanoparticles. FIG. 4, bottom row, shows staining of 4T1 tumors treated with control nanoparticles.

FIG. 5a is a bar graph showing that LyP-1 functionalized nanoparticles target tumors 4-fold greater than nanoparticles fabricated with a control peptide. FIG. 5b is a panel of fluorescent microscopic images showing co-localization of LyP-1 functionalized nanoparticles and tumor associated macrophages in tumors.

FIG. 6 is a panel of microscopic images showing macrophage content by immunohistochemical staining of mouse spleen (top panel), liver (middle panel), and tumor (bottom panel) after in vivo treatment with clodronate-loaded LyP-1-functionalized nanoparticles or PBS saline control.

FIG. 7 is a bar graph quantifying the reduction of tumor associated macrophage density measured by immunohistochemical staining after in vivo treatment with clodronate-loaded LyP-1-functionalized nanoparticles or PBS saline control.

FIG. 8 is a graph showing measured tumor volume after in vivo treatment with clodronate-loaded LyP-1-functionalized nanoparticles, clodronate-loaded ARAL-functionalized nanoparticles, or clodronate only.

### DETAILED DESCRIPTION

Embodiments provided herein relate to compositions including particles that comprise a bisphosphonate compound and a tumor specific targeting peptide or peptidomimetic. In several embodiments, the bisphosphonate compound is clodronate and the tumor specific targeting peptide or peptidomimetic is bound to a surface of the particle. Also, several embodiments related to methods of reducing tumor associated macrophages and methods of treating cancer with compositions are described herein. The compositions described herein were found to successfully target tumor associated macrophages (TAMs) specifically, that thereby reduce the density of TAMs in vitro and in vivo and reduce tumor growth in vivo as measured by tumor volume.

One embodiment is a compound that includes a tumor specific targeting peptide linked to a nanoparticle made of a polymer such as poly(lactic acid-co-glycolic acid) (PLGA). The tumor specific targeting peptide may be the LyP-1 peptide CGNKRTRGC (SEQ ID NO: 1), and the nanoparticle may include a bisphosphonate compound such as clodronate as the effective therapeutic agent.

### Particles

In several embodiments, compositions are provided including a particle which comprises a bisphosphonate compound, such as clodronate. As used herein, the term “particle” refers to a drug delivery vehicle not limited to any size, shape, or dimension, and having a surface to which a tumor specific targeting peptide can be attached and capable of delivering a bisphosphonate compound, such as clodronate.

In some aspects, the particles can include, but is not limited to nanospheres, nanoparticles, microcapsules, nanocapsules, microspheres, microparticles, colloids, aggregates, flocculates, insoluble salts, emulsions and insoluble complexes, any of which can comprise inorganic materials, polymers, polypeptides, proteins, lipids, and surfactants.

### Nanoparticles

In several embodiments, compositions are provided including a nanoparticle which comprises a bisphosphonate compound, such as clodronate. As used herein, the term “nanoparticle” refers to any particle having a greatest dimension (e.g., diameter) that is less than about 2500 nm. In some embodiments, the nanoparticle is a solid or a semi-solid. In some embodiments, the nanoparticle is generally centrosymmetric. In some embodiments, the nanoparticle contains a generally uniform dispersion of solid components.

Nanoparticles can have a characteristic dimension of less than about 1 micrometer, where the characteristic dimension of a particle is the diameter of a perfect sphere having the same volume as the particle. For example, the nanoparticle may have a characteristic dimension that is less than 500 nm, 400 nm, 300 nm, 250 nm, 200 nm, 180 nm, 150 nm, 120 nm, 100 nm, 90 nm, 80 nm, 70 nm, 60 nm, 50 nm, 40 nm, 30 nm, or 20 nm, or any number in between the aforementioned sizes. In some embodiments, the nanoparticle can have a characteristic dimension of 10 nm, 20 nm, 30 nm, 40 nm, 50 nm, 60 nm, 70 nm, 80 nm, 90 nm, 100 nm, 120 nm, 150 nm, 180 nm, 200 nm, 250 nm or 300 nm, or any number in between the aforementioned sizes. In other embodiments, the nanoparticle can have a characteristic dimension of 10-500 nm, 10-400 nm, 10-300 nm, 10-250 nm, 10-200 nm, 10-150 nm, 10-100 nm, 10-75 nm, 10-50 nm, 50-500 nm, 50-400 nm, 50-300 nm, 50-200 nm, 50-150 nm, 50-100 nm, 50-75 nm, 100-500 nm, 100-400 nm, 100-300 nm, 100-250 nm, 100-200 nm, 100-150 nm, 150-500 nm, 150-400 nm, 150-300 nm, 150-250 nm, 150-200 nm, 200-500 nm, 200-400 nm, 200-300 nm, 200-250 nm, 200-500 nm, 200-400 nm or 200-300 nm. Bisphosphonate compounds, such as clodronate, can be incubated with the nanoparticles, and thereby be associated, embedded, encapsulated, loaded, and/or integrated with nanoparticle.

In some embodiments, a composition comprises a population or plurality of nanoparticles, and the population or plurality of nanoparticles can have an average characteristic dimension as described above. A population or plurality of nanoparticles can include at least 20 particles, at least 50 particles, at least 100 particles, at least 300 particles, at least 1,000 particles, at least 3,000 particles, at least 10,000 particles, or greater than 10,000 particles. Various embodiments of the present invention are directed to such populations of particles. For instance, in some embodiments, the particles can each be substantially the same shape and/or size (“monodisperse”). For example, the particles can have a distribution of characteristic dimensions such that no more than about 5% or about 10% of the particles have a characteristic dimension greater than about 10% greater than the average characteristic dimension of the particles, and in some cases, such that no more than about 8%, about 5%, about 3%, about 1%, about 0.3%, about 0.1%, about 0.03%, or about 0.01% have a characteristic dimension greater than about 10% greater than the average characteristic dimension of the particles. In some embodiments, no more than about 5% of the particles have a characteristic dimension greater than about 5%, about 3%, about 1%, about 0.3%, about 0.1%, about 0.03%, or about 0.01% greater than the average characteristic dimension of the particles.

In some embodiments, nanoparticles comprise a material that is biologically inert and can be physiologically tolerated without significant adverse effects by biological systems. Further, a nanoparticle can be comprised of a biodegradable material. It will be understood that there are no restrictions on the physical parameters of a nanoparticle in embodiments provided herein. The physical parameters of a nanoparticle can be optimized, with the desired effect governing the choice of size and shape.

The nanoparticle can comprise a variety of materials including, but not limited to, polymers such as polystyrene, silicone rubber, polycarbonate, polyurethanes, polypropylenes, polymethylmethacrylate, polyvinyl chloride, polyesters, polyethers, and polyethylene.

Additional examples of polymers include, but are not limited to the following: polyethylene glycol (PEG); poly(lactic acid-co-glycolic acid) (PLGA); copolymers of PLGA and PEG; copolymers of poly(lactide-co-glycolide) and PEG; polyglycolic acid (PGA); copolymers of PGA and PEG; poly-L-lactic acid (PLLA); copolymers of PLLA and PEG; poly-D-lactic acid (PDLA); copolymers of PDLA and PEG; poly-D,L-lactic acid (PDLLA); copolymers of PDLLA and PEG; poly(ortho ester); copolymers of poly(ortho ester) and PEG; poly(caprolactone); copolymers of poly(caprolactone) and PEG; polylysine; copolymers of polylysine and PEG; polyethylene imine; copolymers of polyethylene imine and PEG; polyhydroxyacids; polyanhydrides; polyhydroxyalkanoates, poly(L-lactide-co-L-lysine); poly(serine ester); poly(4-hydroxy-L-proline ester); poly-α-(4-aminobutyl)-L-glycolic acid; derivatives thereof; combinations thereof; and copolymers thereof.

Additional examples of polymeric and non-polymeric materials that can be used is several embodiments include, but are not limited to, poly(lactide), poly(hydroxybutyrate), poly(beta-amino) esters and/or copolymers thereof. Alternatively, the particles can comprise other materials, including but not limited to, poly(dienes) such as poly(butadiene) and the like; poly(alkenes) such as polyethylene, polypropylene and the like; poly(acrylics) such as poly(acrylic acid) and the like; poly(methacrylics) such as poly(methyl methacrylate), poly(hydroxyethyl methacrylate), and the like; poly(vinyl ethers); poly(vinyl alcohols); poly(vinyl ketones); poly(vinyl halides) such as poly(vinyl chloride) and the like; poly(vinyl nitriles), poly(vinyl esters) such as poly(vinyl acetate) and the like; poly(vinyl pyridines) such as poly(2-vinyl pyridine), poly(5-methyl-2-vinyl pyridine) and the like; poly(styrenes); poly(carbonates); poly(esters); poly(orthoesters); poly(esteramides); poly(anhydrides); poly(urethanes); poly(amides); cellulose ethers such as methyl cellulose, hydroxyethyl cellulose, hydroxypropyl methyl cellulose and the like; cellulose esters such as cellulose acetate, cellulose acetate phthalate, cellulose acetate butyrate, and the like; poly(saccharides), protein, polypeptides, gelatin, starch, gums, resins and the like. These materials may be used alone, as physical mixtures (blends), or as copolymers.

Several embodiments provided herein relate to compositions including a PLGA nanoparticle containing a bisphosphonate compound, such as clodronate.

Biodegradable, biopolymer (e.g. polypeptides such as BSA, polysaccharides, etc.), other biological materials (e.g. carbohydrates), and/or polymeric compounds are also suitable for use as a nanoparticle scaffold. In various embodiments, the nanoparticle is negatively charged. The nanoparticles may themselves have a negative charge or alternatively a positive charge on them or may be modified to attach a negative charge or positive charge to the scaffold, such as, but not limited to, aldehyde, amine, carboxyl, sulfate, or hydroxyl groups. Factors such as nanoparticle surface charge and hydrophilic/hydrophobic balance of these polymeric materials can be achieved by synthetic modification of the polymers. Such synthetic modification is known in the art and contemplated herein. Various methods for producing the negatively charged nanoparticles are described in U.S. Pat. No. 7,390,384, which is incorporated herein by reference in its entirety.

### Liposomes

In several embodiments, provided are compositions including a liposome which comprises a bisphosphonate compound, such as clodronate. In various embodiments, the bisphosphonate compound is encapsulated inside the liposome. As used herein, the term “liposome” refers to a vesicle composed of amphiphilic lipids arranged in a relatively spherical bilayer or bilayers.

Liposomes are unilamellar or multilamellar vesicles which have a membrane formed from a lipophilic material and an aqueous interior. The aqueous interior portion contains the composition to be delivered. Phospholipids used for liposome formation include, but are not limited to, natural phospholipids such as egg yolk lecithin (phosphatidyl choline), soybean lecithin, lysolecithin, sphingomyelin, phosphatidic acid, phosphatidyl serine, phosphatidyl glycerol, phosphatidyl inositol, phosphatidyl ethanolamine, diphosphatidyl glycerol. Liposome preparation is described, for example, in U.S. Pat. Nos. 7,208,174, 7,108,863, 5,192,549, 6,958,241, and in Ann. Rev. Biophys. Bioeng., 9, 467 (1980), “Liposomes” (Ed. by M. J. Ostro, Marcel Dekker, Inc.) the entire contents of which are incorporated herein by reference. In several embodiments, one or more DNA repair enzyme(s), whether present as a component of an extract or in isolated or purified form, are contained in multilamellar liposomes.

When phospholipids and many other amphipathic lipids are dispersed gently in an aqueous medium they swell, hydrate and spontaneously form multilamellar concentric bilayer vesicles with layers of aqueous media separating the lipid bilayers. These systems commonly are referred to as multilamellar liposomes or multilamellar vesicles (MLV) and usually have diameters of from 0.2 μm to 5 μm. Sonication of MLV results in the formation of small unilamellar vesicles (SUV) with diameters usually in the range of 20 to 100 nm, containing an aqueous solution in the core. Multivesicular liposomes (MVL) differ from multilamellar liposomes in the random, non-concentric arrangement of chambers within the liposome. Amphipathic lipids can form a variety of structures other than liposomes when dispersed in water, depending on the molar ratio of lipid to water, but at low ratios the liposome is the preferred structure.

The physical characteristics of liposomes generally depend on pH and ionic strength. They characteristically show low permeability to ionic and polar substances, but at certain temperatures can undergo a gel-liquid crystalline phase (or main phase) transition dependent upon the physical properties of the lipids used in their manufacture which markedly alters their permeability. The phase transition involves a change from a closely packed, ordered structure, known as the gel state, to a loosely packed, less-ordered structure, known as the liquid crystalline state.

Various types of lipids differing in chain length, saturation, and head group have been used in liposomal formulations for years, including the unilamellar, multilamellar, and multivesicular liposomes mentioned above.

There are at least three types of liposomes. The term “multivesicular liposomes (MVL)” generally refers to man-made, microscopic lipid vesicles comprising lipid membranes enclosing multiple non-concentric aqueous chambers. In contrast, “multilamellar liposomes or vesicles (MLV)” have multiple “onion-skin” concentric membranes, in between which are shell-like concentric aqueous compartments. Multilamellar liposomes and multivesicular liposomes characteristically have mean diameters in the micrometer range, usually from 0.5 to 25 μm. The term “unilamellar liposomes or vesicles (ULV)” generally refers to liposomal structures having a single aqueous chamber, usually with a mean diameter range from about 20 to 500 nm.

Multilamellar and unilamellar liposomes can be made by several relatively simple methods. A number of techniques for producing ULV and MLV are described in the art (for example in U.S. Pat. No. 4,522,803 to Lenk; U.S. Pat. No. 4,310,506 to Baldeschweiler; U.S. Pat. No. 4,235,871 to Papahadjopoulos; U.S. Pat. No. 4,224,179 to Schneider, U.S. Pat. No. 4,078,052 to Papahadjopoulos; U.S. Pat. No. 4,394,372 to Taylor U.S. Pat. No. 4,308,166 to Marchetti; U.S. Pat. No. 4,485,054 to Mezei; and U.S. Pat. No. 4,508,703 to Redziniak).

By contrast, production of multivesicular liposomes generally requires several process steps. Briefly, a common method for making MVL is as follows: The first step is making a “water-in-oil” emulsion by dissolving at least one amphipathic lipid and at least one neutral lipid in one or more volatile organic solvents for the lipid component, adding to the lipid component an immiscible first aqueous component and a biologically active substance to be encapsulated, and optionally adding, to either or both the lipid component and the first aqueous component, an acid or other excipient for modulating the release rate of the encapsulated biologically active substances from the MVL. The mixture is emulsified, and then mixed with a second-immiscible aqueous component to form a second emulsion. The second emulsion is mixed either mechanically, by ultrasonic energy, nozzle atomization, and the like, or by combinations thereof, to form solvent spherules suspended in the second aqueous component. The solvent spherules contain multiple aqueous droplets with the substance to be encapsulated dissolved in them (see Kim et al., Biochem. Biophys. Acta, 728:339-348, 1983). For a comprehensive review of various methods of ULV and MLV preparation, refer to Szoka, et al. Ann. Rev. Biophys. Bioeng. 9:465-508, 1980.

Making multivesicular liposomes can involve inclusion of at least one amphipathic lipid and one neutral lipid in the lipid component. The amphipathic lipids can be zwitterionic, anionic, or cationic lipids. Examples of zwitterionic amphipathic lipids are phosphatidylcholines, phosphatidylethanolamines, sphingomyelins etc. Examples of anionic amphipathic lipids are phosphatidylglycerols, phosphatidylserines, phosphatidylinositols, phosphatidic acids, etc. Examples of cationic amphipathic lipids are diacyl trimethylammoniumpropane and ethyl phosphatidylcholine. Examples of neutral lipids include diglycerides, such as diolein, dipalmitolein, and mixed caprylin-caprin diglycerides; triglycerides, such as triolein, tripalmitolein, trilinolein, tricaprylin, and trilaurin; vegetable oils, such as soybean oil; animal fats, such as lard and beef fat; squalene; tocopherol; and combinations thereof. Additionally, cholesterol or plant sterols can be used in making multivesicular liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

In several embodiments described herein, liposomes that contain one or more DNA repair enzymes, whether present as a component of an extract or in isolated or purified form, can be of various compositions. For example, the liposomes may be made from natural and synthetic phospholipids, glycolipids, and other lipids and lipid congeners; cholesterol, cholesterol derivatives and other cholesterol congeners; charged species which impart a net charge to the membrane; reactive species which can react after liposome formation to link additional molecules to the liposome membrane; and other lipid soluble compounds which have chemical or biological activity.

In various embodiments, liposomes can be composed of phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions can be formed from dimyristoyl phosphatidylglycerol, while anionic fusogenic liposomes can be formed from dioleoyl phosphatidylethanolamine (DOPE). Another type of liposomal composition can be formed from phosphatidylcholine (PC) such as, for example, soybean PC, and egg PC. Another type can be formed from mixtures of phospholipid and/or phosphatidylcholine and/or cholesterol.

Examples of phospholipids suitable for use in several embodiments include but are not limited to DOPC or DC18:1PC=1,2-dioleoyl-sn-glycero-3-phosphocholine; DLPC or DC12:0PC=1,2-dilauroyl-sn-glycero-3-phosphocholine; DMPC or DC14:0PC=1,2-dimyristoyl-sn-glycero-3-phosphocholine; DPPC or DC16:0PC=1,2-dipalmitoyl-sn-glycero-3-phosphocholine; DSPC or DC18:0PC=1,2-distearoyl-sn-glycero-3-phosphocholine; DAPC or DC20:0PC=1,2-diarachidoyl-sn-glycero-3-phosphocholine; DBPC or DC22:0PC=1,2-dibehenoyl-sn-glycero-3-phosphocholine; DC16:1PC=1,2-dipalmitoleoyl-sn-glycero-3-phosphocholine; DC20:1PC=1,2-dieicosenoyl-sn-glycero-3-phosphocholine DC22:1PC=1,2-dierucoyl-sn-glycero-3-phosphocholine; DPPG=1,2-dipalmitoyl-sn-glycero-3-phosphoglycerol; DOPG=1,2-dioleoyl-sn-glycero-3-phosphoglycerol.

Additional examples of phospholipids suitable for use in several embodiments provided herein include but are not limited to those listed in Table 1 below.

TABLE 1

| Abbreviation | CAS | Name | Type |
|---|---|---|---|
| DDPC | 3436-44-0 | 1,2-Didecanoyl-sn-glycero-3- | Phosphatidylcholine |
| | | phosphocholine | |
| DEPA-NA | 80724- | 1,2-Dierucoyl-sn-glycero-3- | Phosphatidic acid |
| | 31-8 | phosphate (Sodium Salt) | |
| DEPC | 56649- | 1,2-Dierucoyl-sn-glycero-3- | Phosphatidylcholine |
| | 39-9 | phosphocholine | |
| DEPE | 988-07-2 | 1,2-Dierucoyl-sn-glycero-3- | Phosphatidylethanolamine |
| | | phosphoethanolamine | |
| DEPG-NA | | 1,2-Dierucoyl-sn-glycero- | Phosphatidylglycerol |
| | | 3[Phospho-rac-(1-glycerol . . . ) | |
| | | (Sodium Salt) | |
| DLOPC | 998-06-1 | 1,2-Dilinoleoyl-sn-glycero-3- | Phosphatidylcholine |
| | | phosphocholine | |
| DLPA-NA | | 1,2-Dilauroyl-sn-glycero-3- | Phosphatidic acid |
| | | phosphate (Sodium Salt) | |
| DLPC | 18194- | 1,2-Dilauroyl-sn-glycero-3- | Phosphatidylcholine |
| | 25-7 | phosphocholine | |
| DLPE | | 1,2-Dilauroyl-sn-glycero-3- | Phosphatidylethanolamine |
| | | phosphoethanolamine | |
| DLPG-NA | | 1,2-Dilauroyl-sn-glycero- | Phosphatidylglycerol |
| | | 3[Phospho-rac-(1-glycerol . . . ) | |
| | | (Sodium Salt) | |
| DLPG-NH4 | | 1,2-Dilauroyl-sn-glycero- | Phosphatidylglycerol |
| | | 3[Phospho-rac-(1-glycerol . . . ) | |
| | | (Ammonium Salt) | |
| DLPS-NA | | 1,2-Dilauroyl-sn-glycero-3- | Phosphatidylserine |
| | | phosphoserine (Sodium Salt) | |
| DMPA-NA | 80724-3 | 1,2-Dimyristoyl-sn-glycero-3- | Phosphatidic acid |
| | | phosphate (Sodium Salt) | |
| DMPC | 18194- | 1,2-Dimyristoyl-sn-glycero-3- | Phosphatidylcholine |
| | 24-6 | phosphocholine | |
| DMPE | 988-07-2 | 1,2-Dimyristoyl-sn-glycero-3- | Phosphatidylethanolamine |
| | | phosphoethanolamine | |
| DMPG-NA | 67232- | 1,2-Dimyristoyl-sn-glycero- | Phosphatidylglycerol |
| | 80-8 | 3[Phospho-rac-(1-glycerol . . . ) | |
| | | (Sodium Salt) | |
| DMPG-NH4 | | 1,2-Dimyristoyl-sn-glycero- | Phosphatidylglycerol |
| | | 3[Phospho-rac-(1-glycerol . . . ) | |
| | | (Ammonium Salt) | |
| DMPG-NH4/NA | | 1,2-Dimyristoyl-sn-glycero- | Phosphatidylglycerol |
| | | 3[Phospho-rac-(1-glycerol . . . ) | |
| | | (Sodium/Ammonium Salt) | |
| DMPS-NA | | 1,2-Dimyristoyl-sn-glycero-3- | Phosphatidylserine |
| | | phosphoserine (Sodium Salt) | |
| DOPA-NA | | 1,2-Dioleoyl-sn-glycero-3- | Phosphatidic acid |
| | | phosphate (Sodium Salt) | |
| DOPC | 4235-95-4 | 1,2-Dioleoyl-sn-glycero-3- | Phosphatidylcholine |
| | | phosphocholine | |
| DOPE | 4004-5- | 1,2-Dioleoyl-sn-glycero-3- | Phosphatidylethanolamine |
| | 1- | phosphoethanolamine | |
| DOPG-NA | 62700- | 1,2-Dioleoyl-sn-glycero- | Phosphatidylglycerol |
| | 69-0 | 3[Phospho-rac-(1-glycerol . . . ) | |
| | | (Sodium Salt) | |
| DOPS-NA | 70614- | 1,2-Dioleoyl-sn-glycero-3- | Phosphatidylserine |
| | 14-1 | phosphoserine (Sodium Salt) | |
| DPPA-NA | 71065- | 1,2-Dipalmitoyl-sn-glycero-3- | Phosphatidic acid |
| | 87-7 | phosphate (Sodium Salt) | |
| DPPC | 63-89-8 | 1,2-Dipalmitoyl-sn-glycero-3- | Phosphatidylcholine |
| | | phosphocholine | |
| DPPE | 923-61-5 | 1,2-Dipalmitoyl-sn-glycero-3- | Phosphatidylethanolamine |
| | | phosphoethanolamine | |
| DPPG-NA | 67232- | 1,2-Dipalmitoyl-sn-glycero- | Phosphatidylglycerol |
| | 81-9 | 3[Phospho-rac-(1-glycerol . . . ) | |
| | | (Sodium Salt) | |
| DPPG-NH4 | 73548- | 1,2-Dipalmitoyl-sn-glycero- | Phosphatidylglycerol |
| | 70-6 | 3[Phospho-rac-(1-glycerol . . . ) | |
| | | (Ammonium Salt) | |
| DPPS-NA | | 1,2-Dipalmitoyl-sn-glycero-3- | Phosphatidylserine |
| | | phosphoserine (Sodium Salt) | |
| DSPA-NA | 108321- | 1,2-Distearoyl-sn-glycero-3- | Phosphatidic acid |
| | 18-2 | phosphate (Sodium Salt) | |
| DSPC | 816-94-4 | 1,2-Distearoyl-sn-glycero-3- | Phosphatidylcholine |
| | | phosphocholine | |
| DSPE | 1069-79-0 | 1,2-Distearoyl-sn-glycero-3- | Phosphatidylethanolamine |
| | | phosphoethanolamine | |
| DSPG-NA | 67232- | 1,2-Distearoyl-sn-glycero- | Phosphatidylglycerol |
| | 82-0 | 3[Phospho-rac-(1-glycerol . . . ) | |
| | | (Sodium Salt) | |
| DSPG-NH4 | 108347- | 1,2-Distearoyl-sn-glycero- | Phosphatidylglycerol |
| | 80-4 | 3[Phospho-rac-(1-glycerol . . . ) | |
| | | (Ammonium Salt) | |
| DSPS-NA | | 1,2-Distearoyl-sn-glycero-3- | Phosphatidylserine |
| | | phosphoserine (Sodium Salt) | |
| Egg Sphingomyelin | | | |
| empty Liposome | | | |
| EPC | | Egg-PC | Phosphatidylcholine |
| HEPC | | Hydrogenated Egg PC | Phosphatidylcholine |
| HSPC | | High purity Hydrogenated Soy PC | Phosphatidylcholine |
| HSPC | | Hydrogenated Soy PC | Phosphatidylcholine |
| LYSOPC | 18194- | 1-Myristoyl-sn-glycero-3- | Lysophosphatidylcholine |
| MYRISTIC | 24-6 | phosphocholine | |
| LYSOPC | 17364- | 1-Palmitoyl-sn-glycero-3- | Lysophosphatidylcholine |
| PALMITIC | 16-8 | phosphocholine | |
| LYSOPC | 19420- | 1-Stearoyl-sn-glycero-3- | Lysophosphatidylcholine |
| STEARIC | 57-6 | phosphocholine | |
| Milk | | 1-Myristoyl-2-palmitoyl-sn- | Phosphatidylcholine |
| Sphingomyelin | | glycero 3-phosphocholine | |
| MPPC | | | |
| MSPC | | 1-Myristoyl-2-stearoyl-sn-glycero- | Phosphatidylcholine |
| | | 3-phosphocholine | |
| PMPC | | 1-Palmitoyl-2-myristoyl-sn- | Phosphatidylcholine |
| | | glycero-3-phosphocholine | |
| POPC | 26853- | 1-Palmitoyl-2-oleoyl-sn-glycero-3- | Phosphatidylcholine |
| | 31-6 | phosphocholine | |
| POPE | | 1-Palmitoyl-2-oleoyl-sn-glycero-3- | Phosphatidylethanolamine |
| | | phosphoethanolamine | |
| POPG-NA | 81490- | 1-Palmitoyl-2-oleoyl-sn-glycero- | Phosphatidylglycerol |
| | 05-3 | 3[Phospho-rac-(1-glycerol) . . . ] | |
| | | (Sodium Salt) | |
| PSPC | | 1-Palmitoyl-2-stearoyl-sn-glycero- | Phosphatidylcholine |
| | | 3-phosphocholine | |
| SMPC | | 1-Stearoyl-2-myristoyl-sn-glycero- | Phosphatidylcholine |
| | | 3-phosphocholine | |
| SOPC | | 1-Stearoyl-2-oleoyl-sn-glycero-3- | Phosphatidylcholine |
| | | phosphocholine | |
| SPPC | | 1-Stearoyl-2-palmitoyl-sn-glycero- | Phosphatidylcholine |
| | | 3-phosphocholine | |

Furthermore, liposomes of the present embodiments can be of various sizes. For example, the diameter of a liposome in various embodiments can be about 300 nm, about 295 nm, about 290 nm, about 285 nm, about 280 nm, about 275 nm, about 270 nm, about 265 nm, about 260 nm, about 255 nm, about 250 nm, about 245 nm, about 240 nm, about 235 nm, about 230 nm, about 225 nm, about 220 nm, about 215 nm, about 210 nm, about 205 nm, about 200 nm, about 195 nm, about 190 nm, about 185 nm, about 180 nm, about 175 nm, about 170 nm, about 165 nm, about 160 nm, about 155 nm, about 150 nm, about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, about 80 nm, about 75 nm, about 70 nm, about 65 nm, about 60 nm, about 55 nm, about 50 nm, about 45 nm, about 40 nm, about 35 nm, about 30 nm, about 25 nm, about 20 nm, about 15 nm, about 10 nm, or about 5 nm. In some embodiments, one or more DNA repair enzyme(s), whether present as a component of an extract or in isolated or purified form, are contained in liposomes that have a diameter of about 200 nm.

Various embodiments include pH sensitive liposomes. Without being bound by theory, it is believed that liposomes which are stable at neutral pH but release their contents at acidic pH can be used to deliver enzymes into the lysozymes of the cytoplasm, whereupon the contents are released. Since many DNA repair enzymes like the T4 endonuclease V are relatively stable at low pH, this method allows efficient delivery of active enzymes into cells.

Liposomes can be made sensitive to the low pH of the lysozymes by the lipid composition. In particular, pH sensitive liposomes can be prepared by using phospholipids which form lipid bilayers when charged but fail to stack in an ordered fashion when neutralized. An example of such a phospholipid is phosphatidylethanolamine, which is negatively charged above pH 9. The net charge of a phospholipid can be maintained at a pH which would otherwise neutralize the head groups by including charged molecules in the lipid bilayer which themselves can become neutralized.

Examples of these charged molecules include but are not limited to oleic acid and cholesteryl hemisuccinate, which are negatively charged at neutral pH but become neutralized at pH 5. The effect of combining these together in a lipid bilayer is that at pH 9 all molecules are charged; at pH 7 the net negative charge of the oleic acid and cholesteryl hemisuccinate maintains the stability of the phosphatidylethanolamine, and at pH 5 all components are protonated and the lipid membrane is destabilized. Additional neutral molecules, such as phosphatidylcholine, can be added to the liposomes as long as they do not interfere with stabilization of the pH sensitive phospholipid by the charged molecules.

By way of example and not limitation, pH sensitive liposomes can be produced by combining phosphatidylethanolamine and cholesteryl hemisuccinate (CHEMS) which destabilizes the liposome at a pH of about less than 4.5. Additionally, inclusion of oleic acid with phosphatidylethanolamine also destabilizes the lipid bilayer at a pH of about less than 6.5, and imparts a net negative charge to the liposome at neutral pH. Liposomes composed of a mixture of phosphatidylcholine and phosphatidylethanolamine are more pH sensitive than those composed of phosphatidylethanolamine alone. In several embodiments, liposomes comprise phospholipids, oleic acid, and cholesterol.

The liposomes of several embodiments described herein can be prepared by combining a phospholipid component with an aqueous component containing the one or more DNA repair enzyme(s), whether present as a component of an extract or in isolated or purified form, under conditions which will result in vesicle formation. The phospholipid concentration should be adequate to form lamellar structures and the aqueous component should be compatible with stability of the DNA repair enzyme(s).

Phospholipids and aqueous components can be combined to form vesicles, for example, by drying the phospholipids onto glass and then dispersing them in the aqueous component; injecting phospholipids dissolved in a vaporizing or non-vaporizing organic solvent into the aqueous component which has previously been heated; and dissolving phospholipids in the aqueous phase with detergents and then removing the detergent by dialysis. The concentration of extract(s) or DNA repair enzyme(s) in the aqueous component can be increased by lyophilizing the extract(s) or DNA repair enzyme(s) onto dried phospholipids and then rehydrating the mixture with a reduced volume of aqueous buffer. Also, methods of producing liposomes in a microfluidizer and adjusting the shear pressure as a means to adjust liposome size are well known in the art.

### Surfactants

Any of the embodiments drawn to compositions including particles, such as nanoparticles or liposomes, can further include one or more surfactants known in the art.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB) The nature of the hydrophilic group (also known as the ‘head’) provides the most useful means for categorizing the different surfactants used in formulations (Rieger, in “Pharmaceutical Dosage Forms,” Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. Popular members of the anionic surfactant class are the alkyl sulfates and the soaps. Also contemplated as examples of anionic surfactants that can be used in several embodiments include stearic acid and sodium behenoyl actylate.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides. The use of surfactants in drug products, formulations and in emulsions has been reviewed (Rieger, in “Pharmaceutical Dosage Forms,” Marcel Dekker, Inc., New York, N.Y., 1988, p. 285). Preferably such surfactants are nonionic and may be in the form of silicones or organic nonionic surfactants.

Suitable silicone surfactants include but are not limited to polyorganosiloxane polymers that have amphiphilic properties, for example contain hydrophilic radicals and lipophilic radicals. These silicone surfactants may be liquids or solids at room temperature. Examples of silicone surfactants that can be used in various embodiments include, but are not limited to: dimethicone copolyols, alkyl dimethicone copolyols, and emulsifying silicone elastomers. Emulsifying silicone elastomers are elastomers that have one or more hydrophilic groups such as hydroxyl, oxyethylene, and the like bonded thereto so as to confer hydrophilic properties to the elastomer. Suitable organic nonionic surfactants may include alkoxylated alcohols or ethers formed by the reaction of an alcohol with a polyalkyleneoxide containing repeating units of alkylene oxide. Preferably, the alcohol is a fatty alcohol having 6 to 30 carbon atoms. Examples of organic nonionic surfactants that can be used in various embodiments include, but are not limited to: steareth 2-100, beheneth 5-30, ceteareth 2-100, ceteareth-25, ceteth 1-45, and the like, which are formed by polyethyleneoxide with the corresponding stearyl/behenyl/cetyl alcohol (wherein the number as used herein designates the number of repeating units of ethylene oxide in the polyethyleneoxide). Other alkoxylated alcohols include esters formed by reaction of polymeric alkylene glycols with glyceryl fatty acid, such as PEG glyceryl oleates, PEG glyceryl stearate; or PEG polyhydroxyalkanotes such as PEG dipolyhydroxystearate wherein the number of repeating ethylene glycol units ranges from 3 to 1000. Nonionic surfactants formed by the reaction of a carboxylic acid with an alkylene oxide or with a polymeric ether are also suitable examples. Monomeric, homopolymeric, or block copolymeric ethers, alkoxylated sorbitan, alkoxylated sorbitan derivatives can also be used as nonionic surfactants in various embodiments.

### Bisphosphonate Compounds

Several embodiments provided herein relate to particles comprising a bisphosphonate compound. The term “bisphosphonate compound” includes all forms thereof including stereoisomers, enantiomers, diastereomers, racemic mixtures and derivatives thereof, for example, salts, acids, esters and the like. Bisphosphonate compounds are synthetic analogues of pyrophosphate (structure P—O—P) in which the central oxygen atom is replaced with a carbon atom and can have a chemical structure represented by the following formula:

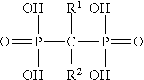

Established nomenclature in the art categorizes bisphosphonates into three generations. The first category comprises the “first-generation” compounds which do not contain a nitrogen atom in their side chains R¹ and R². This category includes, for example, etidronate, clodronate and tiludronate. The secondary category includes the “second-generation” and “third-generation” compounds which contain one or more nitrogen atoms in one of their side chains R¹ or R². Those of the second generation comprise an aliphatic side chain bearing a nitrogen atom or a terminal NH₂ group. Examples include pamidronate, alendronate, ibandronate and neridronate. Those of the third generation bear a heterocyclic nucleus containing a nitrogen atom. Examples include risedronate and zoledronate (imidazole nucleus).

Non-limiting examples of bisphosphonates useful herein include the following: 1-hydroxy-2-(3-pyridinyl)-ethylidene-1,1-bisphosphonic acid (risedronate) as described in U.S. Pat. No. 5,583,122, to Benedict et al., issued Dec. 10, 1996; U.S. Pat. No. 6,410,520 B2, to Cazer et al., issued Jun. 25, 2002; 4-amino-1-hydroxybutylidene-1,1-bisphosphonic acid (alendronic acid or alendronate) as described in U.S. Pat. No. 4,621,077, to Rosini et al., issued Nov. 4, 1986; U.S. Pat. No. 6,281,381 B1, to Finkelstein et al., issued Aug. 28, 2001; U.S. Pat. No. 6,008,207, to Brenner et al., issued Dec. 28, 1999; U.S. Pat. No. 5,849,726, to Brenner et al., issued Dec. 15, 1998; U.S. Pat. Pub. 2001/0021705 A1, by Brenner et al., published Sep. 13, 2001; U.S. Pat. No. 4,922,007, to Kieczykowski et al., issued May 1, 1990; U.S. Pat No. 5,019,651, to Kieczykowski, issued May 28, 1991; 3-amino-1-hydroxypropylidene-1,1-bisphosphonic acid (pamidronate) as described in U.S. Pat. No. 4,639,338, to Stahl et al., issued Jan. 27, 1987; (4-chlorophenyl)thiomethane-1,1-diphosphonic acid (tiludronate) as described in U.S. Pat. No. 4,876,248 to Breliere et al., issued Oct. 24, 1989; 1,1-dichloromethylene-1,1-diphosphonic acid (clodronate) as described in U.S. Pat. No. 3,422,021; cycloheptylaminomethylene-1,1-bisphosphonic acid (cimadronate), as described in U.S. Pat. No. 4,970,335, to Isomura et al., issued Nov. 13, 1990; 1-hydroxy-3-(N-methyl-N-pentylamino)propylidene-1,1-bisphosphonic acid (ibandronate), which is described in U.S. Pat. No. 4,927,814, issued May 22, 1990; 1-hydroxy-2-(imidazol-1-yl)ethane-1,1-bisphosphonic acid (zoledronate); and 1-(N-phenylaminothiocarbonyl)methane-1,1-bisphosphonic acid.

In some embodiments, the bisphosphonate compound is selected from the group consisting of risedronate, alendronate, pamidronate, tiludronate, cimadronate, ibandronate, clodronate, zoledronate, and salts, esters, hydrates, hemihydrates, polymorphs, and solvates thereof, and combinations thereof.

Additional non-limiting examples of bisphosphonate compounds are disclosed in U.S. Patent Application No. 2010/0316676, which is herein incorporated by reference in its entirety.

### Tumor Specific Targeting Peptides or Peptidomimetics

Several embodiments provided herein relate to a particle comprising a tumor specific targeting peptide bound to the surface of the particle. As used herein, the term “tumor specific targeting peptides” refers to a peptide or peptidomimetic that selectively homes or binds to cells associated with a tumor, such but not limited to neoplastic cells and tumor associated macrophages. By “selectively homes or binds” is meant that the peptide or peptidomimetic binds preferentially to tumor cells or tumor associated cells, such as tumor associated macrophages, as compared to normal cells or cells not associated with a tumor. Selective homing or binding generally is characterized by at least a two-fold greater targeting to tumor cells or tumor associated cells (e.g. tumor associated macrophages) compared to control peptides or peptidomimetics. A tumor specific targeting peptide can be characterized by about 2-fold, about 5-fold, about 10-fold, about 20-fold, about 100-fold, or greater preferential targeting to tumor cells or tumor associated cells (e.g. tumor associated macrophages) compared to a control peptide or peptidomimetic. The term “bound” refers to conjugating tumor specific targeting peptides or peptidomimetics to a surface of particles described herein by functionalizing the surface of particles using standard techniques known in the art.

Tumor specific targeting peptides provided herein can have any suitable length, for example, a relatively short length of less than 5, 6, 7, 8, 9, 10, 11, 12, 13, 14, 15, 20, 25, 30, 35 or 40 residues, or any number in between the aforementioned residue lengths. The disclosed peptides also can be useful in the context of a significantly longer sequence. Tumor specific targeting peptides provided herein can also have, for example, a length of up to 50, 100, 150, 200, 250, 300, 400, 500, 1000 or 2000 residues, or any number in between the aforementioned residue lengths. In some embodiments, a tumor specific targeting peptide can have a length of at least 10, 20, 30, 40, 50, 60, 70, 80, 90, 100 or 200 residues. In some embodiments, a peptide can have a length of 5 to 200 residues, 5 to 100 residues, 5 to 90 residues, 5 to 80 residues, 5 to 70 residues, 5 to 60 residues, 5 to 50 residues, 5 to 40 residues, 5 to 30 residues, 5 to 20 residues, 5 to 15 residues, 5 to 10 residues, 10 to 200 residues, 10 to 100 residues, 10 to 90 residues, 10 to 80 residues, 10 to 70 residues, 10 to 60 residues, 10 to 50 residues, 10 to 40 residues, 10 to 30 residues, 10 to 20 residues, 20 to 200 residues, 20 to 100 residues, 20 to 90 residues, 20 to 80 residues, 20 to 70 residues, 20 to 60 residues, 20 to 50 residues, 20 to 40 residues or 20 to 30 residues. As used herein, the term “residue” refers to an amino acid or amino acid analog.

Without being bound by theory, tumor specific targeting peptides bound to the surface of particles provided herein are thought to recognize a receptor expressed on some tumor cells as well as cells associated with such tumor cells, but not relatively expressed in normal tissues. For example, several tumor specific targeting peptides described herein can selectively home or bind to tumor cells and tumor associated macrophages, which originate from circulating monocytes and are recruited into tumors by tumor-derived chemotactic factors.

Various tumor specific targeting peptides are known in the art and can be bound to the surface of a particle in embodiments provided herein. For example, U.S. Patent Publication No. 2007/0149444, the contents of which are herein incorporated by reference in entirety, discloses a peptide or peptidomimetic containing the amino acid sequence CGNKRTRGC (SEQ ID NO: 1) (also known as the LyP-1 peptide) and a peptide or peptidomimetic containing the amino acid sequence GNKRTRG (SEQ ID NO: 2) that home to tumor lymphatic vasculature. In several embodiments, a peptide containing the amino acid of SEQ ID NO: 1 (the LyP-1 peptide) is bound to the surface of a particle. For example, in various embodiments a the surface of a PLGA nanoparticle is functionalized with the LyP-1 peptide by standard crosslinking chemistry known in the art. Without being bound by theory, LyP-1 can target tumor cells and tumor associated macrophages by binding to a protein known as p32 or gClqR receptor expressed both on tumor cells and TAMs in hypoxic or metabolically deprived areas of tumors.

In several embodiments, tumor specific targeting peptides or peptidomimetics can include peptides and peptidomimetics known in the art to target, bind to and/or home to tumor lymphatics, such as lymphatic vessels in and around tumors (which can also be referred to as tumor-associated lymphatic vessels), and/or lymphangiogenic vessels. U.S. Pat. No. 7,919,466 discloses several peptides that target tumor lymphatics and is herein incorporated by reference in its entirety.

For example, in some forms, the amino acid sequence of a peptide that targets tumor lymphatics can be CLSDGK (SEQ ID NO:3), CLSDGK (SEQ ID NO:3) with one, two or three conservative amino acid substitutions, CLSDGK (SEQ ID NO:3) with one non-conservative amino acid substitution, CLSDGK (SEQ ID NO:3) with one non-conservative amino acid substitution and one, two or three conservative amino acid substitutions, or a fragment of any of these forms having at least four consecutive amino acids. Various embodiments can also include an isolated peptide, wherein the peptide comprises an amino acid sequence, wherein the amino acid sequence has at least 70% sequence identity with CLSDGK (SEQ ID NO:3).

As a further example, the amino acid sequence of a peptide that targets tumor lymphatics can be CLSDGKRKC (SEQ ID NO:4), CLSDGKRKC (SEQ ID NO:4) with one, two, three or four conservative amino acid substitutions, CLSDGKRKC (SEQ ID NO:4) with one, two or three non-conservative amino acid substitution, CLSDGKRKC (SEQ ID NO:4) with one, two or three non-conservative amino acid substitution and one, two, three or four conservative amino acid substitutions, or a fragment of any of these forms having at least four consecutive amino acids. Also provided is an isolated peptide, wherein the peptide comprises an amino acid sequence, wherein the amino acid sequence has at least 65% sequence identity with CLSDGKRKC (SEQ ID NO:4).

As a further example, the amino acid sequence of a peptide that targets tumor lymphatics can be CLSDGKPVS (SEQ ID NO:5), CLSDGKPVS (SEQ ID NO:5) with one, two, three or four conservative amino acid substitutions, CLSDGKPVS (SEQ ID NO:5) with one, two or three non-conservative amino acid substitution, CLSDGKPVS (SEQ ID NO:5) with one, two or three non-conservative amino acid substitution and one, two, three or four conservative amino acid substitutions, or a fragment of any of these forms having at least four consecutive amino acids. Also provided is an isolated peptide, wherein the peptide comprises an amino acid sequence, wherein the amino acid sequence has at least 65% sequence identity with CLSDGKPVS (SEQ ID NO:5).

As a further example, the amino acid sequence of a peptide provided herein can be CASLSCR (SEQ ID NO:6), CASLSCR (SEQ ID NO:6) with one, two or three conservative amino acid substitutions, CASLSCR (SEQ ID NO:6) with one or two non-conservative amino acid substitution, CASLSCR (SEQ ID NO:6) with one or two non-conservative amino acid substitution and one, two or three conservative amino acid substitutions, or a fragment of any of these forms having at least four consecutive amino acids. Also provided is an isolated peptide, wherein the peptide comprises an amino acid sequence, wherein the amino acid sequence has at least 65% sequence identity with CASLSCR (SEQ ID NO:6).

As a further example, the amino acid sequence of a peptide provided herein can be CLDGGRPKC (SEQ ID NO:7), CLDGGRPKC (SEQ ID NO:7) with one, two, three or four conservative amino acid substitutions, CLDGGRPKC (SEQ ID NO:7) with one, two or three non-conservative amino acid substitution, CLDGGRPKC (SEQ ID NO:7) with one, two or three non-conservative amino acid substitution and one, two, three or four conservative amino acid substitutions, or a fragment of any of these forms having at least four consecutive amino acids. Also provided is an isolated peptide, wherein the peptide comprises an amino acid sequence, wherein the amino acid sequence has at least 65% sequence identity with CLDGGRPKC (SEQ ID NO:7).

As a further example, the amino acid sequence of the disclosed peptide can be CREAGRKAC (SEQ ID NO:8), CREAGRKAC (SEQ ID NO:8) with one, two, three or four conservative amino acid substitutions, CREAGRKAC (SEQ ID NO:8) with one, two or three non-conservative amino acid substitution, CREAGRKAC (SEQ ID NO:8) with one, two or three non-conservative amino acid substitution and one, two, three or four conservative amino acid substitutions, or a fragment of any of these forms having at least four consecutive amino acids. Also provided is an isolated peptide, wherein the peptide comprises an amino acid sequence, wherein the amino acid sequence has at least 65% sequence identity with CREAGRKAC (SEQ ID NO:8).

As a further example, the amino acid sequence of the disclosed peptide can be CSMSAKKKC (SEQ ID NO:9), CSMSAKKKC (SEQ ID NO:9) with one, two, three or four conservative amino acid substitutions, CSMSAKKKC (SEQ ID NO:9) with one, two or three non-conservative amino acid substitution, CSMSAKKKC (SEQ ID NO:9) with one, two or three non-conservative amino acid substitution and one, two, three or four conservative amino acid substitutions, or a fragment of any of these forms having at least four consecutive amino acids. Also provided is an isolated peptide, wherein the peptide comprises an amino acid sequence, wherein the amino acid sequence has at least 65% sequence identity with CSMSAKKKC (SEQ ID NO:9).

As a further example, the amino acid sequence of the disclosed peptide can be CKTRVSCGV (SEQ ID NO:10), CKTRVSCGV (SEQ ID NO:10) with one, two, three or four conservative amino acid substitutions, CKTRVSCGV (SEQ ID NO:10) with one, two or three non-conservative amino acid substitution, CKTRVSCGV (SEQ ID NO:10) with one, two or three non-conservative amino acid substitution and one, two, three or four conservative amino acid substitutions, or a fragment of any of these forms having at least four consecutive amino acids. Also provided is an isolated peptide, wherein the peptide comprises an amino acid sequence, wherein the amino acid sequence has at least 65% sequence identity with CKTRVSCGV (SEQ ID NO:10).

As a further example, the amino acid sequence of the disclosed peptide can be CAGRRSAYC (SEQ ID NO:11), CAGRRSAYC (SEQ ID NO:11) with one, two, three or four conservative amino acid substitutions, CAGRRSAYC (SEQ ID NO:11) with one, two or three non-conservative amino acid substitution, CAGRRSAYC (SEQ ID NO:11) with one, two or three non-conservative amino acid substitution and one, two, three or four conservative amino acid substitutions, or a fragment of any of these forms having at least four consecutive amino acids. Also provided is an isolated peptide, wherein the peptide comprises an amino acid sequence, wherein the amino acid sequence has at least 65% sequence identity with CAGRRSAYC (SEQ ID NO:11).

As a further example, the amino acid sequence of the disclosed peptide can be CSGGKVLDC (SEQ ID NO:12), CSGGKVLDC (SEQ ID NO:12) with one, two, three or four conservative amino acid substitutions, CSGGKVLDC (SEQ ID NO:12) with one, two or three non-conservative amino acid substitution, CSGGKVLDC (SEQ ID NO:12) with one, two or three non-conservative amino acid substitution and one, two, three or four conservative amino acid substitutions, or a fragment of any of these forms having at least four consecutive amino acids. Also provided is an isolated peptide, wherein the peptide comprises an amino acid sequence, wherein the amino acid sequence has at least 65% sequence identity with CSGGKVLDC (SEQ ID NO:12).

As a further example, the amino acid sequence of the disclosed peptide can be CNRRTKAGC (SEQ ID NO:13), CNRRTKAGC (SEQ ID NO:13) with one, two, three or four conservative amino acid substitutions, CNRRTKAGC (SEQ ID NO:13) with one, two or three non-conservative amino acid substitution, CNRRTKAGC (SEQ ID NO:13) with one, two or three non-conservative amino acid substitution and one, two, three or four conservative amino acid substitutions, or a fragment of any of these forms having at least four consecutive amino acids. Also provided is an isolated peptide, where the peptide comprises an amino acid sequence, wherein the amino acid sequence has at least 65% sequence identity with CNRRTKAGC (SEQ ID NO:13).

As a further example, the amino acid sequence of the disclosed peptide can be CNKRTRGGC (SEQ ID NO:14), CNKRTRGGC (SEQ ID NO:14) with one, two, three or four conservative amino acid substitutions, CNKRTRGGC (SEQ ID NO:14) with one, two or three non-conservative amino acid substitution, CNKRTRGGC (SEQ ID NO:14) with one, two or three non-conservative amino acid substitution and one, two, three or four conservative amino acid substitutions, or a fragment of any of these forms having at least four consecutive amino acids. Also provided is an isolated peptide, where the peptide comprises an amino acid sequence, wherein the amino acid sequence has at least 65% sequence identity with CNKRTRGGC (SEQ ID NO:14).

In several embodiments, tumor specific targeting peptides or peptidomimetics can include peptides and peptidomimetics known in the art to target, bind to and/or home to the vasculature of premalignant or malignant lesions of the pancreas and other organs. U.S. Pat. No. 7,598,341 discloses several peptides that target the vasculature of premalignant or malignant lesions of the pancreas and other organs and is herein incorporated by reference in its entirety.

In several embodiments, tumor specific targeting peptides or peptidomimetics can include peptides and peptidomimetics known in the art to target, bind to and/or home to brain tumors. U.S. Patent Application No. 2011/0130342 discloses peptides that target the vasculature of premalignant or malignant lesions of the pancreas and other organs and is herein incorporated by reference in its entirety.

In several embodiments, tumor specific targeting peptides or peptidomimetics can include peptides and peptidomimetics known in the art to target, bind to and/or home to bladder tumors. U.S. Patent Application No. 2008/0139479 discloses peptides that target the bladder tumors and is herein incorporated by reference in its entirety.

### Synthetic or Non-Naturally Occurring Amino Acids

In several embodiments drawn to a particle comprising a tumor specific targeting peptide bound to the surface of the particle, the tumor specific targeting peptide can comprise synthetic or non-naturally occurring amino acids. Synthetic or non-naturally occurring amino acids refer to amino acids which do not naturally occur in vivo but which, nevertheless, can be incorporated into the peptide structures described herein. Examples of synthetic amino acids are the D-α-amino acids of naturally occurring L-α-amino acid as well as non-naturally occurring D- and L-α-amino acids represented by the formula H₂NCHR⁵COOH where R⁵ is 1) a lower alkyl group, 2) a cycloalkyl group of from 3 to 7 carbon atoms, 3) a heterocycle of from 3 to 7 carbon atoms and 1 to 2 heteroatoms selected from the group consisting of oxygen, sulfur, and nitrogen, 4) an aromatic residue of from 6 to 10 carbon atoms optionally having from 1 to 3 substituents on the aromatic nucleus selected from the group consisting of hydroxyl, lower alkoxy, amino, and carboxyl, 5) -alkylene-Y where alkylene is an alkylene group of from 1 to 7 carbon atoms and Y is selected from the group consisting of (a) hydroxy, (b) amino, (c) cycloalkyl and cycloalkenyl of from 3 to 7 carbon atoms, (d) aryl of from 6 to 10 carbon atoms optionally having from 1 to 3 substituents on the aromatic nucleus selected from the group consisting of hydroxyl, lower alkoxy, amino and carboxyl, (e) heterocyclic of from 3 to 7 carbon atoms and 1 to 2 heteroatoms selected from the group consisting of oxygen, sulfur, and nitrogen, (f) —C(O)R² where R² is selected from the group consisting of hydrogen, hydroxy, lower alkyl, lower alkoxy, and —NR³R⁴ where R³ and R⁴ are independently selected from the group consisting of hydrogen and lower alkyl, (g) —S(O)—R⁶ where n is an integer from 1 to 2 and R⁶ is lower alkyl and with the proviso that R⁵ does not define a side chain of a naturally occurring amino acid.

Other synthetic amino acids include amino acids wherein the amino group is separated from the carboxyl group by more than one carbon atom such as β-alanine, γ-aminobutyric acid, and the like.

Additional synthetic amino acids include, by way of example, the D-amino acids of naturally occurring L-amino acids, L-(1-naphthyl)-alanine, L-(2-naphthyl)-alanine, L-cyclohexylalanine, L-2-aminoisobutyric acid, the sulfoxide and sulfone derivatives of methionine (i.e., HOOC—(H₂NCH)CH₂CH₂—S(O)ₙR⁶) where n and R⁶ are as defined above as well as the lower alkoxy derivative of methionine (i.e., HOOC—(H₂NCH)CH₂CH₂—OR⁶ where R⁶ is as defined above).

These procedures can also be used to synthesize peptides in which amino acids other than the 20 naturally occurring, genetically encoded amino acids are substituted at one, two, or more positions of any of the compounds of various embodiments. For instance, naphthylalanine can be substituted for tryptophan, facilitating synthesis. Other synthetic amino acids that can be substituted into the peptides of the some embodiments include L-hydroxypropyl, L-3,4-dihydroxy-phenylalanyl, d amino acids such as L-d-hydroxylysyl and D-d-methylalanyl, L-α-methylalanyl, β amino acids, and isoquinolyl. D amino acids and non-naturally occurring synthetic amino acids can also be incorporated into the peptides of various embodiments (see, for example, Roberts, et al. 1983 Unusual Amino/Acids in Peptide Synthesis 5:341-449).

One can replace the naturally occurring side chains of the 20 genetically encoded amino acids (or D amino acids) with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclic. In particular, proline analogs in which the ring size of the proline residue is changed from 5 members to 4, 6, or 7 members can be employed. Cyclic groups can be saturated or unsaturated, and if unsaturated, can be aromatic or non-aromatic. Heterocyclic groups preferably contain one or more nitrogen, oxygen, and/or sulphur heteroatoms. Examples of such groups include the furazanyl, furyl, imidazolidinyl, imidazolyl, imidazolinyl, isothiazolyl, isoxazolyl, morpholinyl (for example, morpholino), oxazolyl, piperazinyl (for example, 1-piperazinyl), piperidyl (for example, 1-piperidyl, piperidino), pyranyl, pyrazinyl, pyrazolidinyl, pyrazolinyl, pyrazolyl, pyridazinyl, pyridyl, pyrimidinyl, pyrrolidinyl (for example, 1-pyrrolidinyl), pyrrolinyl, pyrrolyl, thiadiazolyl, thiazolyl, thienyl, thiomorpholinyl (for example, thiomorpholino), and triazolyl. These heterocyclic groups can be substituted or unsubstituted. Where a group is substituted, the substituent can be alkyl, alkoxy, halogen, oxygen, or substituted or unsubstituted phenyl.

One can also readily modify the peptides of various embodiments by phosphorylation (see, for example, W. Bannwarth, et al. 1996 Biorganic and Medicinal Chemistry Letters 6:2141-2146), and other methods for making peptide derivatives of the compounds of various embodiments are described in Hruby, et al. 1990 Biochem J 268:249-262. Thus, the peptide compounds of various embodiments also serve as a basis to prepare peptidomimetics with similar biological activity.

### Terminal Modifications

Those of skill in the art recognize that a variety of techniques are available for constructing peptidomimetics with the same or similar desired biological activity as the corresponding peptide compound but with more favorable activity than the peptide with respect to solubility, stability, and susceptibility to hydrolysis and proteolysis. See, for example, Morgan, et al. 1989 Ann Rep Med Chem 24:243-252. The following describes methods for preparing peptidomimetics modified at the N-terminal amino group, the C-terminal carboxyl group, and/or changing one or more of the amido linkages in the peptide to a non-amido linkage. It is understood that two or more such modifications can be coupled in one peptidomimetic structure (for example, modification at the C-terminal carboxyl group and inclusion of a —CH2-carbamate linkage between two amino acids in the peptide).

1). N-Terminal Modifications

The peptides typically are synthesized as the free acid but, as noted above, could be readily prepared as the amide or ester. One can also modify the amino and/or carboxy terminus of the peptide compounds to produce other useful compounds. Amino terminus modifications include methylation (i.e., —NHCH3 or —NH(CH3)2), acetylation, adding a benzyloxycarbonyl group, or blocking the amino terminus with any blocking group containing a carboxylate functionality defined by RCOO—, where R is selected from the group consisting of naphthyl, acridinyl, steroidyl, and similar groups. Carboxy terminus modifications include replacing the free acid with a carboxamide group or forming a cyclic lactam at the carboxy terminus to introduce structural constraints.

Amino terminus modifications are as recited above and include alkylating, acetylating, adding a carbobenzoyl group, forming a succinimide group, etc. (See, for example, Murray, et al. 1995 Burger's Medicinal Chemistry and Drug Discovery 5th ed., Vol. 1, Manfred E. Wolf, ed., John Wiley and Sons, Inc.) Specifically, the N-terminal amino group can then be reacted as follows:

(a) to form an amide group of the formula RC(O)NH— where R is as defined above by reaction with an acid halide [for example, RC(O)Cl] or symmetric anhydride. Typically, the reaction can be conducted by contacting about equimolar or excess amounts (for example, about 5 equivalents) of an acid halide to the peptide in an inert diluent (for example, dichloromethane) preferably containing an excess (for example, about 10 equivalents) of a tertiary amine, such as diisopropylethylamine, to scavenge the acid generated during reaction. Reaction conditions are otherwise conventional (for example, room temperature for 30 minutes). Alkylation of the terminal amino to provide for a lower alkyl N-substitution followed by reaction with an acid halide as described above will provide for N-alkyl amide group of the formula RC(O)NR—;

(b) to form a succinimide group by reaction with succinic anhydride. As before, an approximately equimolar amount or an excess of succinic anhydride (for example, about 5 equivalents) can be employed and the amino group is converted to the succinimide by methods well known in the art including the use of an excess (for example, ten equivalents) of a tertiary amine such as diisopropylethylamine in a suitable inert diluent (for example, dichloromethane). See, for example, Wollenberg, et al., U.S. Pat. No. 4,612,132 which is incorporated herein by reference in its entirety. It is understood that the succinic group can be substituted with, for example, C2-C6 alkyl or —SR substituents which are prepared in a conventional manner to provide for substituted succinimide at the N-terminus of the peptide. Such alkyl substituents are prepared by reaction of a lower olefin (C2-C6) with maleic anhydride in the manner described by Wollenberg, et al., supra and —SR substituents are prepared by reaction of RSH with maleic anhydride where R is as defined above;

(c) to form a benzyloxycarbonyl-NH— or a substituted benzyloxycarbonyl-NH— group by reaction with approximately an equivalent amount or an excess of CBZ-Cl (i.e., benzyloxycarbonyl chloride) or a substituted CBZ—Cl in a suitable inert diluent (for example, dichloromethane) preferably containing a tertiary amine to scavenge the acid generated during the reaction;

(d) to form a sulfonamide group by reaction with an equivalent amount or an excess (for example, 5 equivalents) of R—S(O)2Cl in a suitable inert diluent (dichloromethane) to convert the terminal amine into a sulfonamide where R is as defined above. Preferably, the inert diluent contains excess tertiary amine (for example, ten equivalents) such as diisopropylethylamine, to scavenge the acid generated during reaction. Reaction conditions are otherwise conventional (for example, room temperature for 30 minutes);

(e) to form a carbamate group by reaction with an equivalent amount or an excess (for example, 5 equivalents) of R—OC(O)Cl or R—OC(O)OC6H4-p-NO2 in a suitable inert diluent (for example, dichloromethane) to convert the terminal amine into a carbamate where R is as defined above. Preferably, the inert diluent contains an excess (for example, about 10 equivalents) of a tertiary amine, such as diisopropylethylamine, to scavenge any acid generated during reaction. Reaction conditions are otherwise conventional (for example, room temperature for 30 minutes); and

(f) to form a urea group by reaction with an equivalent amount or an excess (for example, 5 equivalents) of R—N═C═O in a suitable inert diluent (for example, dichloromethane) to convert the terminal amine into a urea (i.e., RNHC(O)NH—) group where R is as defined above. Preferably, the inert diluent contains an excess (for example, about 10 equivalents) of a tertiary amine, such as diisopropylethylamine. Reaction conditions are otherwise conventional (for example, room temperature for about 30 minutes).

2). C-Terminal Modifications

In preparing peptidomimetics wherein the C-terminal carboxyl group is replaced by an ester (i.e., —C(O)OR where R is as defined above), the resins used to prepare the peptide acids are employed, and the side chain protected peptide is cleaved with base and the appropriate alcohol, for example, methanol. Side chain protecting groups are then removed in the usual fashion by treatment with hydrogen fluoride to obtain the desired ester.

In preparing peptidomimetics wherein the C-terminal carboxyl group is replaced by the amide —C(O)NR3R4, a benzhydrylamine resin is used as the solid support for peptide synthesis. Upon completion of the synthesis, hydrogen fluoride treatment to release the peptide from the support results directly in the free peptide amide (i.e., the C-terminus is —C(O)NH2). Alternatively, use of the chloromethylated resin during peptide synthesis coupled with reaction with ammonia to cleave the side chain protected peptide from the support yields the free peptide amide and reaction with an alkylamine or a dialkylamine yields a side chain protected alkylamide or dialkylamide (i.e., the C-terminus is —C(O)NRR1 where R and R1 are as defined above). Side chain protection is then removed in the usual fashion by treatment with hydrogen fluoride to give the free amides, alkylamides, or dialkylamides.

In another alternative embodiment, the C-terminal carboxyl group or a C-terminal ester can be induced to cyclize by internal displacement of the —OH or the ester (—OR) of the carboxyl group or ester respectively with the N-terminal amino group to form a cyclic peptide. For example, after synthesis and cleavage to give the peptide acid, the free acid is converted to an activated ester by an appropriate carboxyl group activator such as dicyclohexylcarbodiimide (DCC) in solution, for example, in methylene chloride (CH2Cl2), dimethyl formamide (DMF) mixtures. The cyclic peptide is then formed by internal displacement of the activated ester with the N-terminal amine. Internal cyclization as opposed to polymerization can be enhanced by use of very dilute solutions. Such methods are well known in the art.

One can also cyclize the peptides of the invention, or incorporate a desamino or descarboxy residue at the termini of the peptide, so that there is no terminal amino or carboxyl group, to decrease susceptibility to proteases or to restrict the conformation of the peptide. C-terminal functional groups of the compounds of various embodiments include amide, amide lower alkyl, amide di(lower alkyl), lower alkoxy, hydroxy, and carboxy, and the lower ester derivatives thereof, and the pharmaceutically acceptable salts thereof.

### Backbone Modifications

Other methods for making peptide derivatives are described in Hruby, et al. 1990 Biochem J 268(2):249-262, incorporated herein by reference. Thus, the peptide compounds also serve as structural models for non-peptidic compounds with similar biological activity. Those of skill in the art recognize that a variety of techniques are available for constructing compounds with the same or similar desired biological activity as the lead peptide compound but with more favorable activity than the lead with respect to solubility, stability, and susceptibility to hydrolysis and proteolysis. See Morgan, et al. 1989 Ann Rep Med Chem 24:243-252, incorporated herein by reference. These techniques include replacing the peptide backbone with a backbone composed of phosphonates, amidates, carbamates, sulfonamides, secondary amines, and N-methylamino acids.

Peptidomimetics wherein one or more of the peptidyl linkages [—C(O)NH—] have been replaced by such linkages as a —CH₂-carbamate linkage, a phosphonate linkage, a —CH₂-sulfonamide linkage, a urea linkage, a secondary amine (—CH₂NH—) linkage, and an alkylated peptidyl linkage [—C(O)NR⁶— where R⁶ is lower alkyl] are prepared during conventional peptide synthesis by merely substituting a suitably protected amino acid analogue for the amino acid reagent at the appropriate point during synthesis.

Suitable reagents include, for example, amino acid analogues wherein the carboxyl group of the amino acid has been replaced with a moiety suitable for forming one of the above linkages. For example, if one desires to replace a —C(O)NR— linkage in the peptide with a —CH₂-carbamate linkage (—CH₂OC(O)NR—), then the carboxyl (—COOH) group of a suitably protected amino acid is first reduced to the —CH₂OH group which is then converted by conventional methods to a —OC(O)Cl functionality or a para-nitrocarbonate —OC(O)O—C₆H₄-p-NO₂ functionality. Reaction of either of such functional groups with the free amine or an alkylated amine on the N-terminus of the partially fabricated peptide found on the solid support leads to the formation of a —CH₂OC(O)NR— linkage. For a more detailed description of the formation of such —CH₂-carbamate linkages, see Cho, et al. 1993 Science 261:1303-1305.

Similarly, replacement of an amido linkage in the peptide with a phosphonate linkage can be achieved in the manner set forth in U.S. patent application Ser. Nos. 07/943,805, 08/081,577, and 08/119,700, the disclosures of which are incorporated herein by reference in their entirety.

Replacement of an amido linkage in the peptide with a —CH₂-sulfonamide linkage can be achieved by reducing the carboxyl (—COOH) group of a suitably protected amino acid to the —CH₂OH group and the hydroxyl group is then converted to a suitable leaving group such as a tosyl group by conventional methods. Reaction of the tosylated derivative with, for example, thioacetic acid followed by hydrolysis and oxidative chlorination will provide for the —CH2—S(O)₂Cl functional group which replaces the carboxyl group of the otherwise suitably protected amino acid. Use of this suitably protected amino acid analogue in peptide synthesis provides for inclusion of a —CH₂S(O)₂NR— linkage, which replaces the amido linkage in the peptide thereby providing a peptidomimetic. For a more complete description on the conversion of the carboxyl group of the amino acid to a —CH₂S(O)₂Cl group, see, for example, Weinstein, B., 1983 Chemistry & Biochemistry of Amino Acids, Peptides and Proteins Vol. 7, pp. 267-357, Marcel Dekker, Inc., New York, which is incorporated herein by reference.

Replacement of an amido linkage in the peptide with a urea linkage can be achieved in the manner set forth in U.S. patent application Ser. No. 08/147,805 which application is incorporated herein by reference in its entirety.

Secondary amine linkages wherein a —CH₂NH— linkage replaces the amido linkage in the peptide can be prepared by employing, for example, a suitably protected dipeptide analogue wherein the carbonyl bond of the amido linkage has been reduced to a CH₂ group by conventional methods. For example, in the case of diglycine, reduction of the amide to the amine will yield after deprotection H₂NCH₂CH₂NHCH₂COOH which is then used in N-protected form in the next coupling reaction. The preparation of such analogues by reduction of the carbonyl group of the amido linkage in the dipeptide is well known in the art (see, for example, M. W. Remington 1994 Meth Mol Bio 35:241-247).

The suitably protected amino acid analogue is employed in the conventional peptide synthesis in the same manner as would the corresponding amino acid. For example, typically about 3 equivalents of the protected amino acid analogue are employed in this reaction. An inert organic diluent such as methylene chloride or DMF is employed and, when an acid is generated as a reaction by-product, the reaction solvent will typically contain an excess amount of a tertiary amine to scavenge the acid generated during the reaction. One example of a tertiary amine is diisopropylethylamine which is typically employed in about 10-fold excess. The reaction results in incorporation into the peptidomimetic of an amino acid analogue having a non-peptidyl linkage. Such substitution can be repeated as desired such that from zero to all of the amido bonds in the peptide have been replaced by non-amido bonds.

### Methods of Reducing Tumor Associated Macrophage Density

Several embodiments provided herein relate to methods of reducing tumor associated macrophage (TAM) density in a tumor of a subject. As used herein, “subject” includes organisms which are capable of suffering from a cancer treatable with a bisphosphonate compound or who could otherwise benefit from the administration of a bisphosphonate compound as described herein, such as human and non-human animals. Preferred animals include human subjects. The term “non-human animals” of the invention includes all vertebrates, e.g., mammals, e.g., rodents, e.g., mice, rats, and non-mammals, such as non-human primates, e.g., sheep, dog, cow, chickens, amphibians, reptiles, etc. The term “administration” or “administering” includes routes of introducing particles comprising a bisphosphonate compound and a tumor specific targeting peptide or peptidomimetic to a subject to perform their intended function.

TAMs originate from circulating monocytes and their recruitment into tumors is driven by tumor-derived chemotactic factors. TAMs promote tumor cell proliferation and metastasis by secreting a wide range of growth and proangiogenic factors. Consequently, many (but not all) tumors with a high number of TAMs have an increased tumor growth rate, local proliferation and distant metastasis. The extent of TAM infiltration has been used as an inverse prognostic predictor in breast cancer, head and neck cancer, prostate, and uterine cancer (R. D. Leek, R. Landers, S. B. Fox, F. Ng, A. L. Harris, C. E. Lewis, British journal of cancer 1998, 77, 2246; M. R. Young, M. A. Wright, Y. Lozano, M. M. Prechel, J. Benefield, J. P. Leonetti, S. L. Collins, G. J. Petruzzelli, International Journal of Cancer 1997, 74, 69; I. F. Lissbrant, P. Stattin, P. Wikstrom, J. E. Damber, L. Egevad, A. Bergh, International journal of oncology 2000, 17, 445; H. B. Salvesen, L. A. Akslen, International Journal of Cancer 1999, 84, 538). TAMs are also prominent in tumor tissues, comprising up to 80% of the cell mass in breast carcinoma. Also, it is known that bisphosphonate compounds such as clodronate can be used to induce tumor associated macrophage apoptosis.

However, due to their high hydrophilicity and charge, bisphosphonate compounds cannot easily penetrate the cell membrane and thus their effect on non-phagocytic cells is minimal. Without wishing to be bound by theory, it is thought that macrophages take up bisphosphonate compounds by phagocytosis. Accordingly, various methods relate to reducing the density of tumor associated macrophages in a tumor of a subject by administering particles comprising a bisphosphonate compound and a tumor specific targeting peptide or peptidomimetic described herein to a subject.

Accordingly, embodiments relate to the discovery that particles comprising a bisphosphonate compound and a tumor specific targeting peptide or peptidomimetic can reduce the density of macrophages in tumors in vivo in a targeted manner. Several embodiments provided herein relate to methods of reducing tumor associated macrophage density in a tumor of a subject including administering to a subject having a tumor an effective amount of a plurality of particles comprising a bisphosphonate compound and a tumor specific targeting peptide or peptidomimetic as describe herein. As used with respect to reducing tumor associated macrophage density, the term “effective amount” refers to an amount of the particles comprising a bisphosphonate compound and a tumor specific targeting peptide or peptidomimetic sufficient to reduce the density of tumor associated macrophages in the subject. In various embodiments, the effective amount will be sufficient to reduce the density of tumor associated macrophages in a subject from about 5%, 10%, 20%, 25%, 30%, 35%, 40%, 45%, 50%, 55%, 60%, 65%, 70%, 75%, 80%, 85%, 90%, 95%, or 100%, or any number in between the aforementioned percentages. Any assay known in the art can be used to measure tumor associated macrophage density such as immunohistochemical staining of tumor sections using antibodies that specifically detect macrophages.

The effective amount of the “targeted” particles provided herein sufficient to reduce tumor associated macrophage density can be administered in a dosage of about 1 μg/kg, 50 μg/kg, 100 μg/kg, 150 μg/kg, 200 μg/kg, 250 μg/kg, 300 μg/kg, 350 μg/kg, 400 μg/kg, 500 μg/kg, 550 μg/kg, 600 μg/kg, 700 μg/kg, 800 μg/kg, 900 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 75 mg/kg, 100 mg/kg, 125 mg/kg, 150 mg/kg, 175 mg/kg, 200 mg/kg, 225 mg/kg, 250 mg/kg, 275 mg/kg, 300 mg/kg, 325 mg/kg, 350 mg/kg, 375 mg/kg, 400 mg/kg, 425 mg/kg, 450 mg/kg, 425 mg/kg, 450 mg/kg, 475 mg/kg, 500 mg/kg, 750 mg/kg, 1000 mg/kg, or any number in between any two of the aforementioned dosages (mass of particles comprising a bisphosphonate compound and a tumor specific targeting peptide or peptidomimetic/mass of subject).

In another embodiment, the effective amount of the “targeted” particles provided can be administered as a fixed dosage irrespective of the subject's mass. For example, the effective amount of the particles comprising a bisphosphonate compound and a tumor specific targeting peptide or peptidomimetic sufficient to reduce tumor associated macrophage density is a fixed dose of about 1 μg, 50 μg, 75 μg, 100 μg, 150 μg, 200 μg, 250 μg, 300 μg, 350 μg, 400 μg, 500 μg, 550 μg, 600 μg, 700 μg, 800 μg, 900 μg, 1 mg, 5 mg, 10 mg, 15 mg, 20 mg, 25 mg, 30 mg, 35 mg, 40 mg, 45 mg, 50 mg, 75 mg, 100 mg, 200 mg, 300 mg, 400 mg, 500 mg, 600 mg, 700 mg, 800 mg, 900 mg, 1000 mg, 1250 mg, 1500 mg, 1750 mg, 2000 mg, 2250 mg, 2500 mg, 2750 mg, 3000 mg, 3500 mg, 4000 mg, 4500 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, 10,000 mg, or any number in between any two of the aforementioned fixed doses.

### Methods of Treating Cancer

Several embodiments provided herein relate to methods of treating cancer by administering to a subject having cancer an effective amount of a plurality of particles comprising a bisphosphonate compound and a tumor specific targeting peptide or peptidomimetic described herein. As used with respect to treating cancer, the term “effective amount” refers to an amount of particles sufficient to treat cancer, which can be measured by a number of different parameters including, but not limited to, reduction in the size of a tumor in a subject having cancer, reduction in the growth rate or proliferation rate of a tumor in a subject having cancer, preventing metastasis or reducing the extent of metastasis, or extending the survival of a subject having cancer compared to control. In several embodiments, an effective amount of a plurality of particles comprising a bisphosphonate compound and a tumor specific targeting peptide or peptidomimetic described herein will be sufficient to treat cancer in a subject as measured by reduction in the size of a tumor by about 5%, 10%, 20%, 25%, 30%, 35%, 40%, 45%, 50%, 55%, 60%, 65%, 70%, 75%, 80%, 85%, 90%, 95%, or 100%, or any number in between the aforementioned percentages.

The effective amount of the “targeted” particles provided herein sufficient to treat cancer in a subject can be administered in a dosage of about 1 μg/kg, 50 μg/kg, 100 μg/kg, 150 μg/kg, 200 μg/kg, 250 μg/kg, 300 μg/kg, 350 μg/kg, 400 μg/kg, 500 μg/kg, 550 μg/kg, 600 μg/kg, 700 μg/kg, 800 μg/kg, 900 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 75 mg/kg, 100 mg/kg, 125 mg/kg, 150 mg/kg, 175 mg/kg, 200 mg/kg, 225 mg/kg, 250 mg/kg, 275 mg/kg, 300 mg/kg, 325 mg/kg, 350 mg/kg, 375 mg/kg, 400 mg/kg, 425 mg/kg, 450 mg/kg, 425 mg/kg, 450 mg/kg, 475 mg/kg, 500 mg/kg, 750 mg/kg, 1000 mg/kg, or any number in between any two of the aforementioned dosages (mass of particles comprising a bisphosphonate compound and a tumor specific targeting peptide or peptidomimetic/mass of subject).

In another embodiment, the effective amount of the “targeted” particles provided can be administered as a fixed dosage irrespective of the subject's mass. For example, the effective amount of the particles comprising a bisphosphonate compound and a tumor specific targeting peptide or peptidomimetic sufficient to reduce the size of a tumor in a subject is a fixed dose of about 1 μg, 50 μg, 75 μg, 100 μg, 150 μg, 200 μg, 250 μg, 300 μg, 350 μg, 400 μg, 500 μg, 550 μg, 600 μg, 700 μg, 800 μg, 900 μg, 1 mg, 5 mg, 10 mg, 15 mg, 20 mg, 25 mg, 30 mg, 35 mg, 40 mg, 45 mg, 50 mg, 75 mg, 100 mg, 200 mg, 300 mg, 400 mg, 500 mg, 600 mg, 700 mg, 800 mg, 900 mg, 1000 mg, 1250 mg, 1500 mg, 1750 mg, 2000 mg, 2250 mg, 2500 mg, 2750 mg, 3000 mg, 3500 mg, 4000 mg, 4500 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, 10,000 mg, or any number in between any two of the aforementioned fixed doses.

As contemplated herein, examples of cancers that can be treated by administering particles comprising a bisphosphonate compound and a tumor specific targeting peptide or peptidomimetic described herein include, but are not limited to melanoma, epithelial cancer, squamous cell cancer, small cell lung cancer, non-small cell lung cancer, glioma, hepatocellular (liver) carcinoma, thyroid tumor, a leukemia, gastric (stomach) cancer, prostate cancer, breast cancer, ovarian cancer, bladder cancer, lung cancer, glioblastoma, endometrial cancer, kidney cancer, colon cancer, ovarian cancer, uterine cancer, hepatocellular cancer, pancreatic cancer, esophageal carcinoma, head and neck cancers, esophageal cancer, lymphoma, mesothelioma, sarcomas, carcinomas, biliary (cholangiocarcinoma), small bowel adenocarcinoma, pediatric malignancies and epidermoid carcinoma.

### Administration and Pharmaceutical Forms

The particles comprising a bisphosphonate compound and a tumor specific targeting peptide or peptidomimetic can be administered in a variety of ways and pharmaceutical forms in the embodiments provided herein for reducing tumor associated macrophage density or treating cancer. As such, provided herein are several compositions drawn to pharmaceutical compositions comprising the particles including a bisphosphonate compound and a tumor specific targeting peptide or peptidomimetic described herein and a pharmaceutically acceptable carrier or diluent depending on the route and form of administration.

Examples of routes of administration that may be used include injection (subcutaneous, intravenous, parenterally, intraperitoneally, intrathecal), or oral routes. The pharmaceutical preparations may be given by forms suitable for each administration route. For example, these preparations can be administered in tablets or capsule form, by injection or orally. The injection can be bolus or can be continuous infusion. The particles comprising a bisphosphonate compound and a tumor specific targeting peptide or peptidomimetic can be administered alone, or in conjunction with either another agent or agents known in the art for treating cancer or with a pharmaceutically-acceptable carrier, or both.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN, polyethylene glycol (PEG).

The compositions may be in the “pharmaceutical form” of tablets, capsules, powders, granules, lozenges, liquid or gel preparations. Tablets and capsules for oral administration may be in a form suitable for unit dose presentation and may contain conventional excipients. Examples of these are: binding agents such as syrup, acacia, gelatin, sorbitol, tragacanth, and polyvinylpyrrolidone; fillers such as lactose, sugar, maize-starch, calcium phosphate, sorbitol or glycine; tableting lubricants, such as magnesium stearate, silicon dioxide, talc, polyethylene glycol or silica; disintegrants, such as potato starch; or acceptable wetting agents, such as sodium lauryl sulfate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, e.g., sorbitol, syrup, methyl cellulose, glucose syrup, gelatin, hydrogenated edible fats, emulsifying agents, e.g., lecithin, sorbitan monooleate, or acacia; non-aqueous vehicles (including edible oils), e.g., almond oil, fractionated coconut oil, oily esters such as glycerine, propylene glycol, or ethyl alcohol; preservatives such as methyl or propyl p-hydroxybenzoate or sorbic acid, and, if desired, conventional flavoring or coloring agents.

Solid dosage forms for oral administration include capsules, tablets, pills, powders, and granules. In such solid dosage forms, the particles comprising a bisphosphonate compound and a tumor specific targeting peptide or peptidomimetic can be admixed with at least one inert pharmaceutically acceptable carrier such as sucrose, lactose, or starch. Such dosage forms can also comprise, as is normal practice, additional substances other than inert diluents, for example, lubricating agents such as magnesium stearate. In the case of capsules, tablets, and pills, the dosage forms may also comprise buffering agents. Tablets and pills can additionally be prepared with enteric coatings known in the art.

Liquid dosage forms for oral administration include pharmaceutically acceptable emulsions, solutions, suspensions, syrups, with the elixirs containing inert diluents commonly used in the art, such as water. Besides such inert diluents, compositions can also include adjuvants, such as wetting agents, emulsifying and suspending agents, and sweetening, flavoring, and perfuming agents.

The particles comprising a bisphosphonate compound and a tumor specific targeting peptide or peptidomimetic can also be administered parenterally. The phrases “parenteral administration” and “administered parenterally” as used herein includes, for example, modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. Parenteral administration can include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

For parenteral administration, the peptides can be, for example, formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils may also be used. The vehicle or lyophilized powder may contain additives that maintain isotonicity (for example, sodium chloride, mannitol) and chemical stability (for example, buffers and preservatives). The formulation is sterilized by commonly used techniques. For example, a parenteral composition suitable for administration by injection is prepared by dissolving 1.5% by weight of active ingredient in 0.9% sodium chloride solution.

The pharmaceutical compositions described herein can be administered as a single dose or in multiple doses; administered either as individual therapeutic agents or in combination with other therapeutic agents; and combined with conventional therapies, which may be administered sequentially or simultaneously.

While the present invention has been described in some detail for purposes of clarity and understanding, one skilled in the art will appreciate that various changes in form and detail can be made without departing from the true scope of the invention.

### EXAMPLES

Having generally described embodiments drawn to particles comprising a bisphosphonate compound and a tumor specific targeting peptide or peptidomimetic, and methods for reducing tumor associated macrophage density in a tumor of a subject and methods of treating cancer in a subject by administering said particles, a further understanding can be obtained by reference to certain specific examples which are provided herein for purposes of illustration only, and are not intended to be limiting.

### Example 1

### Preparation of Nanoparticles Comprising Clodronate and Tumor Specific Targeting Peptide

Clodronate was purchased from Sigma. Sulfo-SMCC was from Pierce. Biodegradable PLGA (50:50 lactide:glycolide, MW 47,000) was purchased from Boehringer Ingelheim. The organic solvent dichloromethane (DCM) and poly(vinylalcohol) (PVA, MW 9000-10,000, 80% hydrolyzed) were also purchased from Sigma.

Peptides were synthesized with automatic microwave assisted peptide synthesizer (Liberty; C E M, Matthews) using standard solid-phase Fmoc/t-Bu chemistry. During synthesis, the peptides were labeled with 5(6)-carboxyfluorescein (FAM) with a 6-aminohexanoic acid spacer separating the dye from sequence.

A water-in-oil-in-water double-emulsion-solvent evaporation method was used to prepare clodronate-loaded PLGA NPs (E. Cohen-Sela, O. Rosenzweig, J. Gao, H. Epstein, I. Gati, R. Reich, H. D. Danenberg, G. Golomb, J Control Release 2006, 113, 23). Briefly, 0.5 ml of a 4% aqueous solution of clodronate (pH adjusted to 7.4) was emulsified with 3 ml of 3% PLGA solution in DCM using a probe sonicator at 50 W power for 60 s. The primary emulsion was then added to 20 ml of 2% PVA solution containing CaCl2 as 2:1 molar ratio of Ca:clodronate and emulsified at 50 W power for 90 s using a probe sonicatior. DCM was evaporated by magnetic stirring for 4 h at 4° C. Particles were collected by centrifugation and washed 3× with DDW, lypholized and stored at 4° C. until further use.

Amine-modified PLGA polymer for LyP-1 peptide (amino acid sequence: CGNKRTRGC; SEQ ID N0:1) conjugation was prepared by conjugating hexaethylene glycol-diamine to the carboxylic terminal group of PLGA-COOH using the protocols as described in Yoon et. al. Biomaterials 2004, 25, 5613. Amine functionalized PEGylated PLGA nanoparticles were then prepared by using the double-emulsion-solvent-evaporation method as described above and using a 50:50 blend of PLGA-PEG and PLGA-NH2.

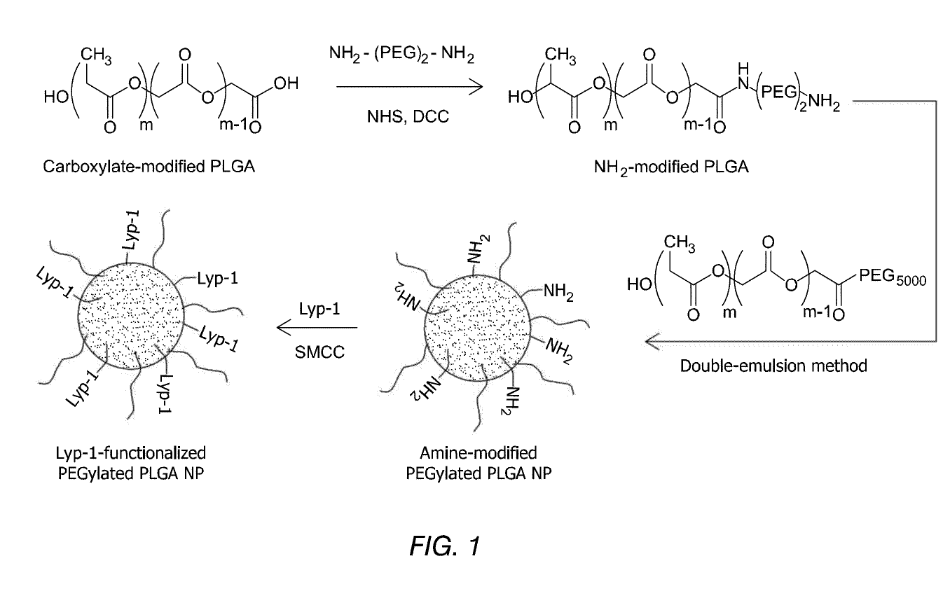

As illustrated in FIG. 1, LyP-1-functionalized PLGA NPs were prepared by coupling the LyP-1 peptide to amine-functionalized PLGA NPs through the peptide cysteine sulfhydryl group using a sulfo-SMCC cross-linker. Briefly, 20 mg NPs were suspended in 700 μl of PBS and 300 μl from 10 mM sulfo-SMCC was added. After incubation at room temperature for 30 minutes, the excess cross-linker was removed by filtration through a Nap-10 column (GE Healthcare).

LyP-1 peptide labeled with FAM was dissolved in sterile nitrogen purged water and added to the PLGA-sulfo-SMCC conjugate in small aliquots over a period of 1 hour. The peptide was used at twofold molar excess relative to PLGA. Excess peptide was removed by repeated centrifugation. Control NPs were prepared similarly except that LyP-1 was replaced with FAM-X-Cys (X refers to 6-aminohexanoic acid spacer).

### Example 2

### Clodronate Nanoparticle Formulation

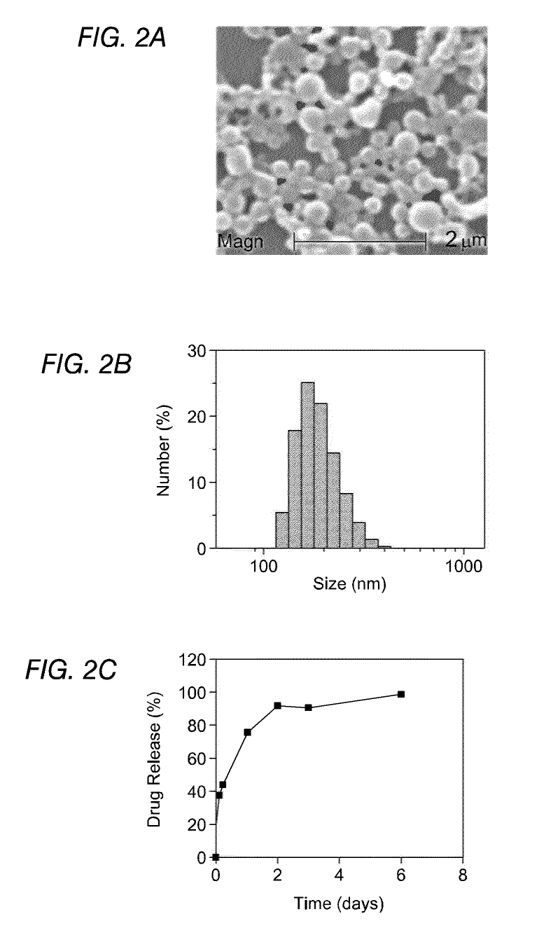

The clodronate-loaded PLGA nanoparticles prepared according to Example 1 were studied for examined encapsulation efficiency (EE), morphology, particle size distribution and release kinetics. During the study, morphology of the nanoparticles was studied through scanning electron microscopy. As seen from the electron micrographs in FIG. 2a, the nanoparticles were smooth and spherical in shape. The size distribution illustrated in FIG. 2b shows an average diameter of 180±20 nm.

An analysis of the amount of clodronate encapsulated in the NPs revealed that 51% of the drug was entrapped in the particles. The release rate of entrapped clodronate was studied and the result is shown in FIG. 2c. The kinetic data showed that majority of the drug release occurred in the first day (approximately 80%) and the release amount almost remained unchanged after 48 h. The “burst effect” release phenomenon, where drug is released in the very early stages, was observed as almost 50% of the drug was released in 4 h. This release phenomenon is typical for a hydrophilic drug such as clodronate encapsulated using the w/o/w emulsion technique due the rapid migration of the water soluble drug towards the external aqueous phase.

### Example 3

### Selective Toxicity of Nanoparticles Comprising Clodronate and Tumor Specific Targeting Peptide

The cytotoxicity of clodronate alone and clodronate-loaded PLGA nanoparticles (clodNP) as prepared in Example 1 were tested in vitro on mouse macrophage (RAW264.7) and human fibroblast (CCL-110) cell lines. The RAW264.7 monocyte/macrophage mouse cell line was obtained from the American Type Culture Collection (ATCC) and cultured in DMEM media containing L-glutamine and sodium pyruvate (Mediatech), supplemented with 10% fetal bovine serum (FBS) and 100 IU/ml penicillin/streptomycin.

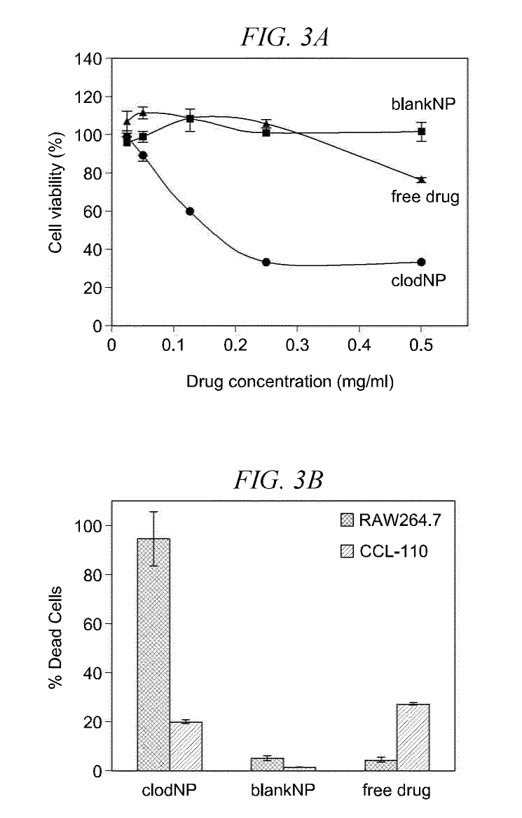

Cells were incubated with clodNPs as well as with blank NPs and free clodronate drug at a concentration of 250 μg/ml. As shown in FIG. 3a, clodNPs but not blank NPs exhibited dose-dependent toxicity of RAW264.7 macrophages in vitro. Furthermore, as shown in FIG. 3a, clodNPs exhibited substantially greater toxicity of RAW264.7 macrophages in vitro compared to clodronate alone. As shown in FIG. 3b, treatment of RAW264.7 macrophages in vitro with clodNPs resulted in an 8-fold increase in cell death compared to blank NPs and free drug. clodNPs showed little cytotoxicity towards human fibroblasts cell line (CCL-110) used as control.

### Example 4

### Selective Targeting of Tumor Associated Macrophages

To test the efficacy of nanoparticles conjugated with LyP-1 peptide for tumor “homing”, the PLGA nanoparticles conjugated with FAM-labeled LyP-1 (LyP-1-NPs) described in Example 1 were injected into Balb/C mice bearing 4T1 tumors. Briefly, 4T1 cells were maintained in Dulbecco's modified eagle's medium supplemented with 10% FBS and 1% glutamine penicillin-streptomycin at 37° C. and 5% CO2. To induce 4T1 tumors, female Balb/c mice were injected with 2×106 4T1 cells in PBS into the mammary fat pad region. Two weeks after tumor cell injection LyP-1-NPs and control NPs were injected (15 mg/kg animal weight in 200 μl PBS) via the tail vein. The mice were sacrificed 3 hours post injection by cardiac perfusion under anesthesia, and organs were dissected and analyzed for particle homing. Tissues were processed and immunohistochemical analysis was performed as described previously (P. P. Karmali, V. R. Kotamraju, M. Kastantin, M. Black, D. Missirlis, M. Tirrell, E. Ruoslahti, Nanomedicine 2009, 5, 73).

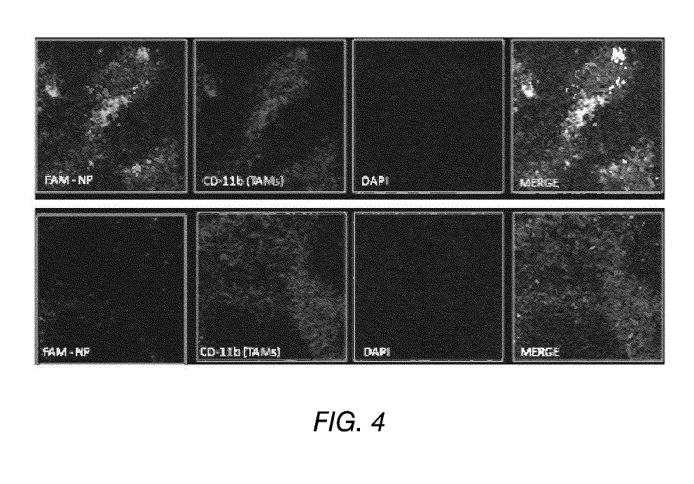

As shown in FIG. 4, LyP-1-NPs (FIG. 4, top row) showed enhanced accumulation in extravascular tumor tissue compared to non-functionalized control NPs (FIG. 4, bottom row). Nanoparticles, nuclei, and CD-11b+ tumor associated macrophages (TAMs) were fluorescently stained as shown.

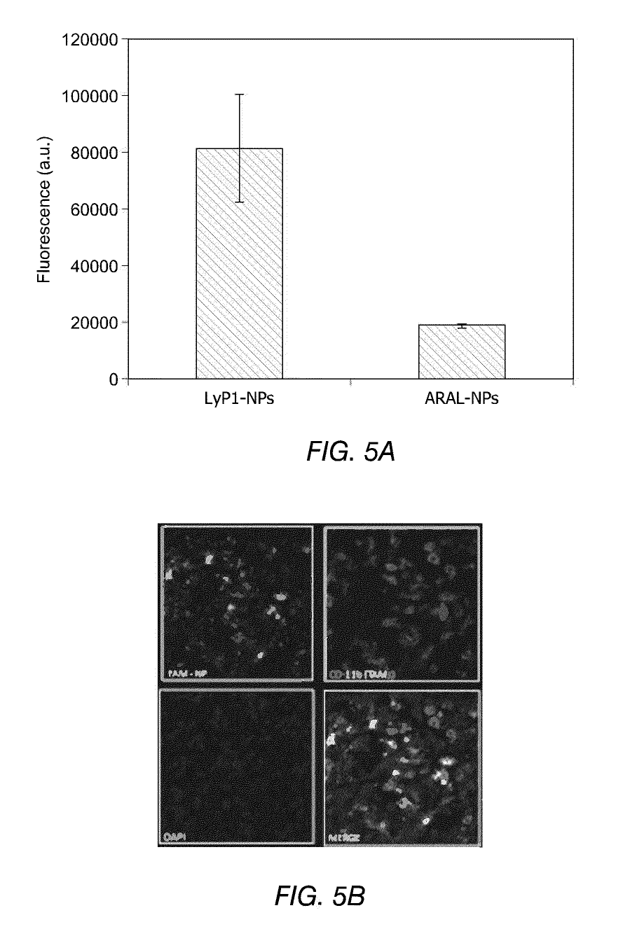

As shown in FIG. 5a, a 4-fold increase in nanoparticle targeting to tumors was observed with LyP-1-NPs compared to nanoparticles functionalized with the control peptide ARAL (ARAL-NPs). As shown in FIG. 5b, LyP-1-NPs co-localize with tumor associated macrophages (TAMs) in tumors.

### Example 5

### Reduction of Tumor Associated Macrophage Density in Vivo

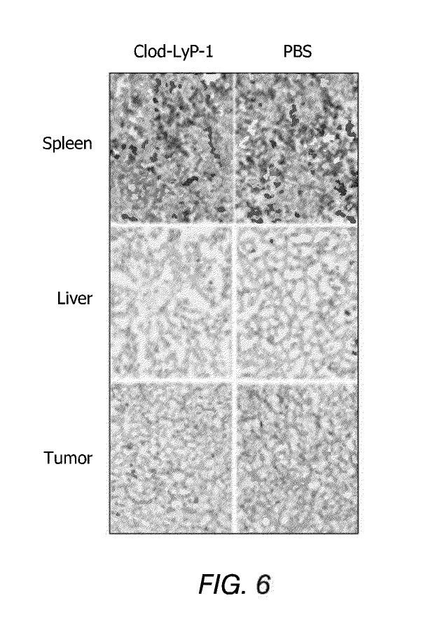

Clodronate-loaded LyP-1-functionalized PLGA nanoparticles (Clod-LyP-1) as prepared in Example 1 were administered to Balb/C mice bearing 4T1 tumors according to the methodology described in Example 4. As shown in FIG. 6, tissues from spleen (FIG. 6, top row), liver (FIG. 6, middle row), and tumor (FIG. 6, bottom row) were processed and immunohistochemical analysis using an anti-F4/80 antibody was performed to visualize macrophage content before and after treatment with Clod-LyP-1 NPs or PBS control.

As shown in FIG. 6, fewer tumor associated macrophages were observed in tumors treated with Clod-LyP-1 NPs compared to PBS control. However, a similar staining pattern of non-tumor associated macrophages in the spleen and liver were observed with treatment with Clod-LyP-1 NPs and PBS control, suggesting that Clod-LyP-1 NPs specifically target and reduce tumor associated macrophages.

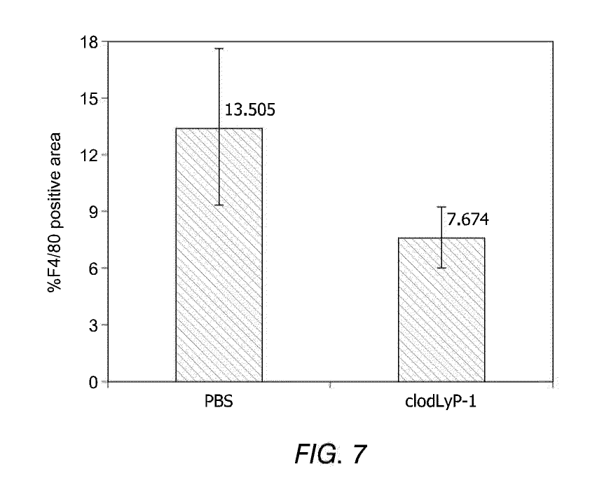

As shown in FIG. 7, a 43% reduction in tumor associated macrophage density (measured by F4/80 staining) was observed in tumors of mice injected with Clod-LyP-1 NPs compared to PBS control.

### Example 6

### Reduction of Tumor Growth in Vivo

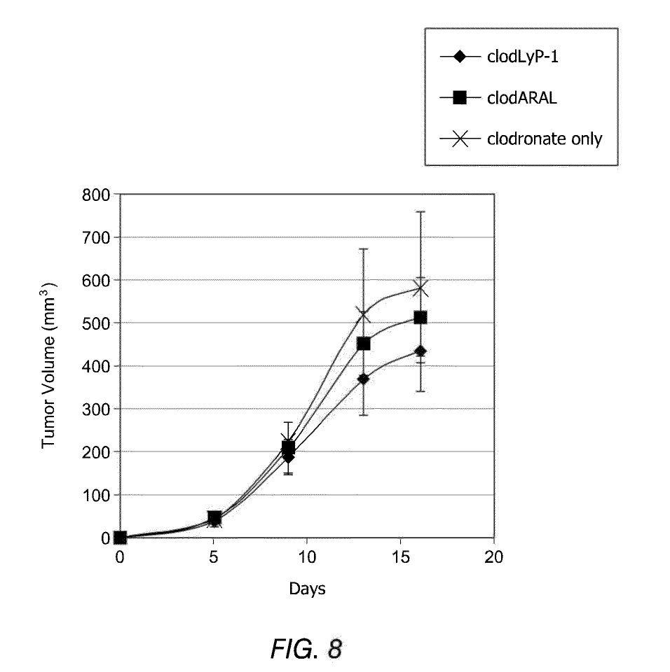

Clodronate-loaded LyP-1-functionalized PLGA nanoparticles (Clod-LyP-1) as prepared in Example 1, clodronate-loaded control peptide ARAL-functionalized PLGA nanoparticles (Clod-ARAL), or clodronate alone were administered to Balb/C mice bearing 4T1 tumors according to the methodology described in Example 4. As shown in FIG. 8, a smaller tumor volume was observed at the indicated days after treatment with Clod-LyP-1 compared to treatment with controls Clod-ARAL and clodronate alone.

### Example 7

### Treatment of a Patient Having Cancer

A patient is diagnosed with a cancerous tumor. The patient is administered a composition comprising particles that include a bisphosphonate compound and a tumor specific targeting peptide or peptidomimetic. The composition is administered to the patient intravenously at a dosage of about 0.1 to 10,000 mg of composition/kg body weight as determined by the attending physician.

The patient is monitored for reduction in the size of a tumor in the subject, reduction in tumor growth, prevention or reduction of metastasis, and/or extension of survival of the subject compared to control patients. It is discovered that the composition is useful for treating the cancerous tumor.

What is claimed is:

1. A composition comprising a particle which comprises a bisphosphonate compound and a tumor specific targeting peptide or peptidomimetic bound to a surface on the particle.

2. The composition of claim 1, wherein the particle is a nanoparticle.

3. The composition of claim 2, wherein the nanoparticle comprises a polymer.

4. The composition of claim 3, wherein the polymer is poly(lactide-co-glycolide) (PLGA).

5. The composition of claim 4, wherein the bisphosphonate compound is clodronate.

6. The composition of claim 5, wherein the peptide or peptidomimetic comprises the amino acid sequence of SEQ ID NO: 1.

7. The composition of claim 1, wherein the particle is a liposome.

8. The composition of claim 7, wherein the bisphosphonate compound is clodronate.

9. The composition of claim 8, wherein the peptide or peptidomimetic comprises the amino acid sequence of SEQ ID NO: 1.

10. A method of reducing tumor associated macrophage density in a tumor of a subject comprising administering to a subject having a tumor an effective amount of a plurality of particles comprising a bisphosphonate compound and a tumor specific targeting peptide or peptidomimetic bound to a surface on the particles, wherein the effective amount of the particles is sufficient to reduce the density of tumor associated macrophages in the tumor of the subject.

11. The method of claim 10, wherein the particle is a nanoparticle comprising a polymer.

12. The method of claim 11, wherein the polymer comprises poly(lactide-co-glycolide) (PLGA).

13. The method of claim 12, wherein the bisphosphonate compound is clodronate.

14. The method of claim 13, wherein the peptide or peptidomimetic comprises the amino acid sequence of SEQ ID NO: 1.

15. A method of treating cancer in a subject, comprising:
administering to a subject having cancer an effective amount of a plurality of particles comprising a bisphosphonate compound and a tumor specific targeting peptide or peptidomimetic bound to a surface on the particles, wherein the effective amount of the particles is sufficient to treat the cancer.

16. The method of claim 15, wherein the particle is a liposome.

17. The method of claim 15, wherein the particle is a nanoparticle comprising a polymer.

18. The composition of claim 15, wherein the polymer is poly(lactide-co-glycolide) (PLGA).

19. The method of claim 18, wherein the bisphosphonate molecule is clodronate.

20. The method of claim 19, wherein the peptide or peptidomimetic comprises the amino acid sequence of SEQ ID NO: 1.
